(19) United States
(12) Patent Application Publication
(10) Pub. No.: US 2021/0295968 A1
(43) Pub. Date: Sep. 23, 2021

(54) SYSTEMS, DEVICES AND METHODS FOR SECURING, STORING AND ACCESSING PET AND VETERINARY INFORMATION

(71) Applicant: Deirdre Kamber Todd, Allentown, PA (US)

(72) Inventor: Deirdre Kamber Todd, Allentown, PA (US)

(21) Appl. No.: 17/337,111

(22) Filed: Jun. 2, 2021

Related U.S. Application Data
(63) Continuation of application No. 16/408,140, filed on May 9, 2019, now abandoned.

(51) Int. Cl. G16H 10/65; G06F 21/62

(52) U.S. Cl. CPC G16H 10/65; G06F 21/32; G06F 21/6245

(57) ABSTRACT

A system for providing medical information for a pet includes a pet medical information form that contains medical information about a pet, a pet medical information passport that includes identifying and medical information about the pet, and a pet tag worn by the pet, the pet tag including a OR code or a near field communication (NFC) device that stores emergency contact information for the pet and medical and insurance information for the pet.

### CROSS-REFERENCE TO RELATED APPLICATIONS

The present patent application is a continuation of U.S. patent application Ser. No. 16/408,140, filed on May 9, 2019, the disclosure of which is hereby incorporated by reference herein.

### BACKGROUND OF THE INVENTION

### Field of the Invention

The present patent application is generally related to maintaining medical records, and is more particularly related to secure systems, devices, and methods for storing and accessing medical and identification information for patients, dependents, and pets.

### Description of the Related Art

Many of the items that are used for identification purposes have been reduced in size to provide greater accessibility and compactness. For example, pocket-sized identification cards have been developed that are used for drivers' licenses, medical insurance cards, health club memberships, and bank and credit cards. Recently, many individuals have begun to use their cell phones and smart phones for providing payments, authentication and gaining access to secure locations.

In spite of the above advances, when emergencies arise, many individuals are totally unprepared to respond, and lack immediate access to the types of information that are needed by health care providers to provide identification information, advanced directives and comprehensive medical treatment. For example, most individuals do not carry emergency contact information. Moreover, if an individual is incapacitated or lacks full awareness, they have no means by which to provide emergency responders, doctors, or other health care providers with immediate access to relatives who can serve as health care decision-makers, or to other emergency information such as drug allergies, living wills and protected health information permissions.

The above-noted situations are further complicated by the advent of federal and state health care privacy laws, which mandate completing many forms in order to obtain comprehensive health care for physical and mental conditions. This means that many individuals are confronted with a sea of paperwork they do not understand when they need to focus of their own health or the health of a loved one. Moreover, as the health care privacy laws prevent automatic dissemination of information to the majority caretakers, such as adult children, and grant automatic dissemination of health information to undesirable recipients, such as former spouses, individuals require a process by which they can identify with whom their medical information should, and should not, be shared.

In recent years, the pre-treatment information that must be provided to doctors and hospitals has changed drastically. In the past, a pre-treatment check-in would typically include a quick review of an individual's insurance coverage. Now, however, the pre-treatment review requires a drawn-out process for satisfying privacy regulations, photo identification cards, authorizations for accessing medical records, multiple insurance cards, advanced directives (in cases of admission to hospitals) and complicated legal reviews regarding who has been granted access to medical records based upon pre-event authorizations and designations.

Many of the forms that must be signed by patients and medical personnel are required by federal law under the Health Insurance Portability and Accessibility Act of 1996 (HIPAA) in addition to state law privacy laws; all health care providers must adhere to the requirements of the Act, as well as the rules and regulations pertaining to, and be in compliance with the standards set out pursuant to 45 C.F.R. §§ 160 and 164.

In many instances, patients and family members are required to sign release and authorization forms without reviewing and/or fully understanding the legal and medical consequences of the act of signing. This is because the forms are lengthy and contain complicated language that is not easy to understand, especially during stressful or emergency situations.

As complex as health care policies and forms are, these processes and requirements are even more complicated when a patient is incapacitated or unconscious, or if the patient is a minor child or dependent adult for whom a parent or family member (i.e., a caretaker) is required to sign documents to act in the patients best interests. The caretaker of the patient may be cut off from the decisions because there is no ready proof of the power to act on the patients behalf. As the HIPAA law provides automatic disclosure to select few family members by law, it does not provide access to most caretakers without the patient's formal authorization. As a result, most caretakers, such as parents of adult children or caretakers for the elderly, cannot access the protected health information of their charges if the patient cannot formally consent. Moreover, as the HIPAA law provides for no means to restrict authorization to a recipient who is entitled to a patient's information by law, no legal mechanism exists to prevent the dissemination of individuals' protected health information from disclosure. Thus, in cases where spouses are separated or where family members are unwelcome, HIPAA provides no means to prevent doctors from improperly sharing a patient's health information, even with a signed HIPAA authorization.

While HIPAA, as a federal law, provides a baseline for medical information sharing, many states have enacted state laws that are more stringent than the federal standards. Especially in cases relating to mental health records, drug or alcohol treatment, or highly sensitive ailments, such as HIV/AIDS, state laws often require additional permissions for patient information sharing, an authorization that cannot be shared when a patient is unable to express his or her intents.

In a patient's preferred doctor's office, the staff may know the circumstances of responsibility for a minor child or a caregiver to an older patient, and, in many instances, legal requirements have often been previously shared with the doctor's office. During an emergency, however, when care needs to be provided in a timely fashion by any available doctor, hospital or emergency medical team, the patient may not be able to provide the necessary information about a caregiver, a close relative, a guardian, or another person to contact in case of emergency, or to provide access to other medical records that may help in providing emergency treatment, and the accompanying caregiver may be prevented from assisting medical personnel because the guardian/caretaker cannot prove authorization to make medical decisions or to be granted access to the medical records of the patient.

Travel has also become a significant issue in patient information sharing. Whether traveling within or outside the United States, individuals are at greater risk for the inaccessibility of their medical information, should a medical issue arise. Moreover, with the challenges posed by language barriers and differing laws outside of the United States, medical information authorization becomes far more problematic.

Another tragic circumstance that often occurs involves missing persons. Almost every day there is sad news of a person gone missing. Children are abducted, both by strangers and by someone they know, including parents, Young people run away, are kidnapped or make risky choices. Adults simply disappear. Individuals with Alzheimer's or dementia frequently wander. Substance abuse, mental illness and unfamiliar surroundings can affect all persons. No one can imagine their loved one disappearing, but every missing person has a family and friends who cannot imagine life without them.

Experts say that the first 48 hours after a person is lost or missing are the most crucial. The sooner a missing person's full information is available to authorities, the more likely it is that the missing person will be found. Yet, in many instances, obtaining a person's full scope of information, including appearance, fingerprints and DNA samples, can waste valuable hours if the information is not readily available. Both physical and biological descriptions of a missing person can immediately aid officials in their search and recovery. In addition, once a missing person is found, he or she may require immediate medical attention, which includes knowing any current medical conditions of the person, understanding the habits of an individual, such as drug use, or knowing who to contact to obtain the medical background of the individual.

In view of the above-noted deficiencies, there is a need for improved systems, devices and methods for storing medical information and providing easy access to the biologics, personal identification information, and medical information when it is needed by patients, medical personnel, family members and guardians.

While the immediate availability of personal and medical information is critical to a patient and his or her caretaker, contrivances must be in place to protect the access to such information by unintended recipients. Both as a function of law and for the wellbeing of the patient, it is essential that information only reach those providers on a need to know basis.

Concerns of safety and medical information are not limited to our human charges. Nearly seventy percent of American families own one or more pet. Many people treat their pets as family members. With the vast increase in therapy and service dog usage in the United States moreover, many individuals formally rely on their pets for their own personal wellbeing. Although methods such as metal tags and microchip embedding have become means for locating a pet, these systems are slow, laborious, and provide little essential information for the wellbeing of the animal beyond the name of the animal and a home location. As pets are often found with injuries or in critical health, moreover, these means for location do not provide the necessary veterinary information in a timely fashion.

While medical services are provided by both doctors and veterinarians, the systems used by each generally are not identical. Medical identity theft has become a serious concern in the United States since the advent of computers. Due largely to medical identity theft, the HIPAA law was created in 1996 and continues to evolve to protect patients against unlawful dissemination of protected health information. HIPAA's protections only extend to humans, not to pets. As a result, doctors, hospitals and emergency medical services generally have far more secure electronic systems than those found in veterinary offices and hospitals. Due to these concerns, most health care systems for humans only permit contactless technology in-house, such as an NFC field and QR code, and do not permit the use of Universal Serial Bus (“USB”) devices for the sharing of information, due to concerns of misuse and virus dissemination. Veterinarians will generally allow the use of USB devices for the storing and sharing of pet information.

### SUMMARY OF THE INVENTION

In one embodiment, a system for providing medical information preferably includes a medical information passport having a near field communication (NFC) tag that stores access to emergency contact information. In one embodiment, the NFC may store a command to send a message to an emergency contact, e.g., “text phone #555-5555 with the following message: Susie is receiving emergency medical care. Please call me back at this telephone number.”

In one embodiment, the system desirably includes a protective sleeve for storing the medical information passport. In one embodiment, the protective sleeve is made of a material (e.g., metal, aluminum) that blocks radio waves to prevent unauthorized access of the emergency contact information stored on the NFC tag. In one embodiment, the protective sleeve may include one or more metal films.

In one embodiment, the medical information passport is assigned to an individual, such as a patient, a child, or a dependent adult (e.g., Alzheimer's patient). In one embodiment, the medical information passport may be assigned to a pet such as a dog or a cat.

In one embodiment, the medical information passport preferably contains medical information that is printed thereon and that is used for providing medical care for the individual. In one embodiment, the printed medical information may include photographs, personal identifying information, treating physician information, medical conditions, medications, emergency contacts, advanced directives information, and/or HIPAA information.

In one embodiment, the medical information passport includes a paper substrate, an NFC tag or chip, and a transparent film covers the paper substrate and the NFC tag/chip. The paper substrate and the transparent film may be made of recycled materials, and the transparent film may be biodegradable.

In one embodiment, the command information stored on the NFC tag desirably includes one telephone number for an emergency contact. During an emergency situation, a first responder may scan the NFC tag to send a text message from his or her cellular telephone to a predetermined emergency contact to notify the emergency contact that the individual is undergoing emergency medical treatment. The first responder may also add to the text to include other important information, such as his or her contact information or the intended hospital location.

In one embodiment, the medical information passport may include a QR code that links the medical information passport with a computer database having electronically stored emergency treatment information for the individual assigned to the medical information passport. In one embodiment, the electronically stored emergency treatment information is not password protected.

In one embodiment, the medical information passport may include a QR code that links the medical information passport with a computer database having electronically stored office check-in data for the individual assigned to the medical information passport. In one embodiment, the electronically stored office check-in data is password protected on a HIPAA compliant, secure website.

In one embodiment, the medical information passport may include a QR code that links the medical information passport with a computer database having electronically stored medical records for the individual assigned to the medical information passport. In one embodiment, the electronically stored medical records are password protected on a HIPAA compliant, secure website.

In one embodiment, a single OR code on the medical information passport will provides a link to the computer database that contains the emergency treatment information, the office check-in data, and the medical records so that all of the information that is stored on the computer database may be accessed using the single OR code.

In one embodiment, the electronically stored medical records may include MRI images, X-ray images, lab test results, doctor's notes, pharmacy records, insurance records, government records, surgical records, pharmacy records, advanced directives, and/or any records provided by the individual assigned to the medical information passport.

In one embodiment, a system for providing medical information desirably includes a medical information passport assigned to an individual. In one embodiment, the medical information passport desirably includes printed medical information that is used for providing medical care for the individual, a near field communication (NFC) tag that stores emergency contact information for notifying an emergency contact, and a OR code that links the medical information passport with a computer database having electronically stored medical information associated with the individual. In one embodiment, the system may include a protective sleeve for storing the medical information passport. In one embodiment, the protective sleeve preferably includes a material that blocks radio waves to prevent unauthorized access of the emergency contact information stored on the NFC tag. In one embodiment, the protective sleeve may include one or more metal films that are adapted to block radio waves to prevent unauthorized NFC skimming of the NFC tag.

In one embodiment, the electronically stored medical information preferably includes electronically stored emergency treatment information that is customized for the individual, electronically stored office check-in data that is customized for the individual, and electronically stored medical records that are customized for the individual. The quantity and type of customized information that is entered into the computer database may be controlled by the individual, caretaker, guardian or parent of the individual.

In one embodiment, the printed medical information may include photographs, personal identifying information, guardianship documents, fingerprint scans, treating physician information, medical conditions, medications, emergency contacts, advanced directives information, and/or HIPAA information.

In one embodiment, the medical information passport may include a paper substrate, an NFC chip, and a transparent film covering the paper substrate. The paper substrate and the transparent film may be made of recycled materials, and the transparent film may be biodegradable.

In one embodiment, the printed medical information identifies a location on the medical information passport for scanning the NFC tag and may includes an editable message or instructions that scanning the NFC tag will send a text message to an emergency contact.

In one embodiment, the emergency contact information stored on the NFC tag desirably includes at least one telephone number for the emergency contact.

In one embodiment, a system for providing medical information may include a medical information passport assigned to an individual, whereby the medical information passport includes a paper substrate and a transparent film overlying the paper substrate.

In one embodiment, the system may include medical information printed on the paper substrate that is customized for the individual and that is used for identifying and providing medical care for the individual, and a near field communication (NFC) tag coupled with the paper substrate, whereby the NFC tag has emergency contact information stored therein including a telephone number for an emergency contact.

In one embodiment, a QR code desirably overlies the paper substrate. The QR code may link the medical information passport with a computer database having electronically stored medical information that is customized for the individual.

In one embodiment, a protective sleeve is adapted for storing the medical information passport. In one embodiment, the protective sleeve desirably includes a material that blocks radio waves to prevent unauthorized access of the emergency contact information stored on the NFC tag.

In one embodiment, a system for storing and accessing medical information preferably includes a portable device, such as a medical information passport, that is carried by an individual or patient. In one embodiment, the portable device may be configured to provide a methodology for accessing medical information and medical records. The portable devices may include a wide range of mechanisms including but not limited to cards, credit cards, clip-on tools, tags, wrist bands, bracelets, watches, and key-chains, which may all be carried by or maintained under the control of an individual or patient.

In one embodiment, the medical information stored on the portable device, or accessed using the portable device, may include: 1) medical check-in information used by a doctor, medical personnel, or a hospital; 2) emergency information used by a first responder, an emergency medical technician (EMT), or emergency room personnel; 3) a personal medical record clearinghouse for the patient or a caretaker; 4) authorization forms for releasing health information pursuant to the Health Insurance Portability and Accountability Act of 1996 (HIPAA) to those individuals who have been granted access to the health information, and to deny access to those individuals who should not have access to the health information; 5) immediate notification of an emergency to an individual's emergency contact(s); and 6) identification information for those who are minors, children, dependents, dependent adults (e.g., dementia patients), individuals traveling abroad, and/or pets.

In one embodiment, the system for accessing medical information disclosed herein may be modified to serve different types of customers. In one embodiment, the portable system may include a medical information passport, referred to as a MediPass, for individuals and patients who wish to have control over their own medical information.

In one embodiment, the medical information passport provides basic medical information, such as name, address, date of birth, blood type, primary physician, primary physician phone number, medical conditions, emergency contact information, advanced directives, and HIPAA Authorizations. There is no information included on the medical information passport, such as social security numbers, which would be of use to the average pickpocket.

In one embodiment, the medical information passport preferably includes a near field communication (NFC) device or tag incorporated therein, which may be scanned using a smart phone or other device. Upon scanning the NFC device, the scanning smart phone will generate an automatic, but editable, text message from the user's phone that is sent to a phone number of an emergency contact previously identified by the owner of the medical information passport.

In one embodiment, the medical information passport preferably includes a OR code that may be scanned using a smart phone or an electronic device that is programmed to read OR codes. In an emergency situation, without requiring a password, scanning the OR code will enable first responders or hospitals to obtain emergency information for treating the individual who owns the medical information passport. In non-emergency situations, in conjunction with providing a password for accessing the HIPAA compliant secure website, scanning the QR code will enable medical personnel to obtain office check-in information and the individual's medical records.

In one embodiment, the medical information passport is preferably stored in a protective sleeve that has a metal lining, which prevents the NFC device/tag in the medical information passport from being illegally scanned by a third party until the medical information passport is removed from the protective sleeve. Thus, the protective sleeve prevents unauthorized access to the NFC tag and the information stored on the NFC tag.

In one embodiment, a medical information passport may include a USB drive that provides a link that is used to access a pet's identification information, emergency information and veterinary records via cloud computing, The pet's identification and emergency information is not password protected. The pet's veterinary records are encrypted and accessing the information requires a password.

In one embodiment, individuals using the medical information passport may also utilize a keychain and/or a bracelet as a backup. In one embodiment, the keychain may glow in the dark, and may include a OR Code or an NFC tag, which perform as described above for the medical information passport, In one embodiment, a bracelet may have an NFC device or tag that provides access to the carrier's check-in information, emergency files, and/or medical records. All embodiments for access to patient information use storage on a HIPAA compliant, secure website.

In one embodiment, a system for accessing medical information may include a dependent medical information passport, referred to as a CoPass, which is utilized by individuals who seek to have and control identity and medical information for dependents (e.g., a minor, a child, a dependent adult), or for an individual who wants to share theft medical information with another (e.g., a partner, an adult roommate, a friend, or a family member other than a spouse).

In one embodiment, the dependent medical information passport has the same design and features that are present in the medical information passport.

In one embodiment, the system may include an emergency kit for use with dependents and people at risk of disappearing (e.g., teens, adults with dementia). In one embodiment, the emergency kit may include a DNA sample swab pack, and a fingerprint kit, the results of which are scanned into a medical information file associated with the dependent.

In one embodiment, the dependent emergency kit may include a magnetic sheet that may include the following types of information for a child or a dependent: a photo ID, indications of identifying marks (such as glasses, tattoos, etc.), address, emergency contact(s), emergency health information, and access to the dependent's online files through a OR code.

In one embodiment, the dependent medical information passport may include a keychain having an NFC tag and/or OR code as described herein, a bracelet having an NFC device/tag or a OR code as described herein, and a OR Code screensaver for smartphones.

In one embodiment, a system for accessing medical information may include a pet medical information passport, referred to as a PetPass, which is preferably used by the owners of pets. The pet medical information passport may include a pet tag, such as a glow-in-the-dark tag with an NFC device/tag that is designed to text a message to the owner of a lost pet. In one embodiment, a pet tag may include a OR Code, which may be scanned to use a link to access an online pet form that may provide additional information about the pet, such as medical information, veterinarian, adverse drug interactions, allergies, etc.

In one embodiment, the portable system for pets may include a pet information sheet having a magnetic backing for being secured to a metal surface (e.g., a refrigerator door). The pet information sheet preferably includes a photograph of the animal, description and critical information that may be accessed if the animal gets lost or needs emergent medical care; it also allows the owner to print out the information sheet as a sign, on a home computer, if the pet should go missing.

in one embodiment, the portable systems for accessing medical information disclosed herein enables operators to access different types of medical information. Different security protocols may be adhered to for accessing different types of medical information. In one embodiment, the medical information may be stored remotely, may be accessed via the internet, may be accessed via secure websites, may be accessed via secure servers, and may incorporate Cloud computing storage systems that provide security and fully comply with HIPAA requirements.

in one embodiment, the medical check-in information does not require a password for accessing the information. In one embodiment, the medical check-in information may include one or more of the following: driver's license number, insurance cards, insurance carrier information, HIPAA Authorization, HIPAA Non-Release of personal health information, power of attorney, and/or living will information.

In one embodiment, the emergency information does not require a password to access the information. In one embodiment, the emergency information may include one or more of the following: blood type, emergency contacts, patient contact information, preferred doctors, preferred hospitals, power of attorney, living will, organ donation, allergies, prior surgeries, medications taken, insurance Information, HIPAA Authorization statement, and/or HIPAA Non-Release of personal health information.

In one embodiment, a system for accessing medical information preferably requires a password to access medical records. In one embodiment, the medical records file may include one or more of the following: all records provided by the client, MRI images, X-ray images, lab test results, and/or all other medical documents.

These and other preferred embodiments of the present patent application will be described in more detail below.

### BRIEF DESCRIPTION OF THE DRAWINGS

FIG. 1 is a first page of a form used for collecting medical information, in accordance with one embodiment of the present patent application.

FIG. 2 is a second page of a form used for collecting medical information, in accordance with one embodiment of the present patent application.

FIG. 3 is a third page of a form used for collecting medical information, in accordance with one embodiment of the present patent application.

FIG. 4 is a fourth page of a form used for collecting medical information, in accordance with one embodiment of the present patent application.

FIG. 5 is a first page of a form used for authorizing the release of health information pursuant to the Health Insurance Portability and Accountability Act of 1999 (HIPAA), in accordance with one embodiment of the present patent application.

FIG. 6 is a second page of a form used for authorizing the release of health information pursuant to the Health Insurance Portability and Accountability Act of 1999 (HIPAA), in accordance with one embodiment of the present patent application.

FIG. 7 is a perspective view of a protective sleeve containing a medical information passport, in accordance with one embodiment of the present patent application.

FIG. 8 is a top view of the protective sleeve and the medical information passport of FIG. 7 after the medical information passport has been removed from the protective sleeve, in accordance with one embodiment of the present patent application.

FIG. 9 shows the outer surfaces of the first and second halves of the medical information passport of FIG. 8, in accordance with one embodiment of the present patent application.

FIG. 10 shows the outer surface of he first half of the medical information passport shown in FIG. 9.

FIG. 11 shows the outer surface of the second half of the medical information passport shown in FIG. 9.

FIG. 12 shows inner surfaces of the first and second halves of he medical information passport shown in FIG. 9.

FIG. 13 shows the inner surface of the first half of the medical information passport shown in FIG. 12.

FIG. 14 shows the inner surface of the second half of the medical information passport shown in FIG. 12.

FIG. 15 is a perspective view of a bracelet containing medical information, in accordance with one embodiment of the present patent application.

FIG. 16 is a top view of a watch containing medical information, in accordance with one embodiment of the present patent application.

FIG. 17 is a perspective view of the watch of FIG. 16 in communication with a computer system for linking the watch with medical information for a patient that is stored on the computer system, in accordance with one embodiment of the present patent application.

FIG. 18 is a form used for collecting medical information for a dependent, in accordance with one embodiment of the present patent application.

FIG. 19 shows cotton swabs, an ink pad, and a collection pouch used for collecting medical information for a dependent, in accordance with one embodiment of the present patent application.

FIG. 20 shows a medical information passport for a dependent and a protective sleeve configured to hold the medical information passport, in accordance with one embodiment of the present patent application.

FIG. 21 shows a perspective view of outer surfaces of the first and second halves of the medical information passport of FIG. 20, in accordance with one embodiment of the present patent application.

FIG. 22 shows the outer surface of the first half of the medical information passport of FIG. 21.

FIG. 23 shows the outer surface of the second half of the medical information passport of FIG. 21.

FIG. 24 shows a perspective view of inner surfaces of the first and second halves of the medical information passport of FIG. 20, in accordance with one embodiment of the present patent application.

FIG. 25 shows the inner surface of the first half of the medical information passport of FIG. 24.

FIG. 26 shows the inner surface of the second half of the medical information passport of FIG. 24.

FIG. 27 shows a medical information sheet that displays medical and identifying information for a dependent, in accordance with one embodiment of the present patent application.

FIG. 28 shows the medical information sheet of FIG. 27 having a magnetic backing for being secured to a metallic surface, in accordance with one embodiment of the present patent application.

FIG. 29 shows a medical information form used to collect medical information for a pet, in accordance with one embodiment of the present patent application.

FIG. 30A shows a front face of a medical information passport for a pet, in accordance with one embodiment of the present patent application.

FIG. 30B shows a rear face of a medical information passport for a pet, in accordance with one embodiment of the present patent application.

FIG. 31A shows the front face of the medical information passport of FIGS. 30A and 30B including a USB flash drive swung into an extended position, in accordance with one embodiment of the present patent application.

FIG. 31B shows the rear face of the medical information passport of FIGS. 30B and 31A with the USB flash drive in the extended position.

FIG. 32A shows a front face of a pet tag that provides a link to medical information for a pet, in accordance with one embodiment of the present patent application.

FIG. 32B shows a rear face of the pet tag shown in FIG. 32A.

FIG. 33A shows a front face of a pet tag that provides a link to medical information for a pet, in accordance with one embodiment of the present patent application.

FIG. 33B shows a rear face of the pet tag shown in FIG. 33A.

FIG. 34 shows a medical information sheet that displays medical information for a pet, in accordance with one embodiment of the present patent application.

FIG. 35A shows a front view of a protective folder configured to hold a medical information passport, in accordance with one embodiment of the present patent application.

FIG. 35B shows a top perspective view of the protective folder of FIG. 35A with a medical information passport stored in the protective folder, in accordance with one embodiment of the present patent application.

FIG. 36 shows a top view of the protective folder of FIGS. 35A and 35B with the medical information passport stored in the second half of the protective folder, in accordance with one embodiment of the present patent application.

FIG. 37 shows a top view of the second half of the protective folder of FIG. 36 after the medical information passport has been removed from the second half, in accordance with one embodiment of the present patent application.

FIG. 38 is a front view of outer surfaces of the s and second halves of the protective folder shown in FIGS. 35A and 35B.

FIG. 39 is a front view of the outer surface of the first half of the protective folder shown in FIG. 38.

FIG. 40 is a front view of the outer surface of the second half of the protective folder shown in FIG. 38.

FIG. 41 is a front view of inner surfaces of the first and second halves of the protective folder shown in FIGS. 35A and 35B.

FIG. 42 is a front view of the inner surface of the first half of the protective folder shown in FIG. 41.

FIG. 43 is a front view of the inner surface of the second half of the protective folder shown in FIG. 41.

FIG. 44A shows a front face of a medical information passport, in accordance with one embodiment of the present patent application.

FIG. 44B shows a rear face of the medical information passport shown in FIG. 44A.

FIG. 45A shows the front face of the medical information passport of FIGS. 44A and 44B with a USB flash drive rotated into an extended position, in accordance with one embodiment of the present patent application.

FIG. 458 shows the rear face of the medical information passport of FIGS. 44A and 448 with the USB flash drive rotated into the extended position, in accordance with one embodiment of the present patent application.

### DETAILED DESCRIPTION OF PREFERRED EMBODIMENTS

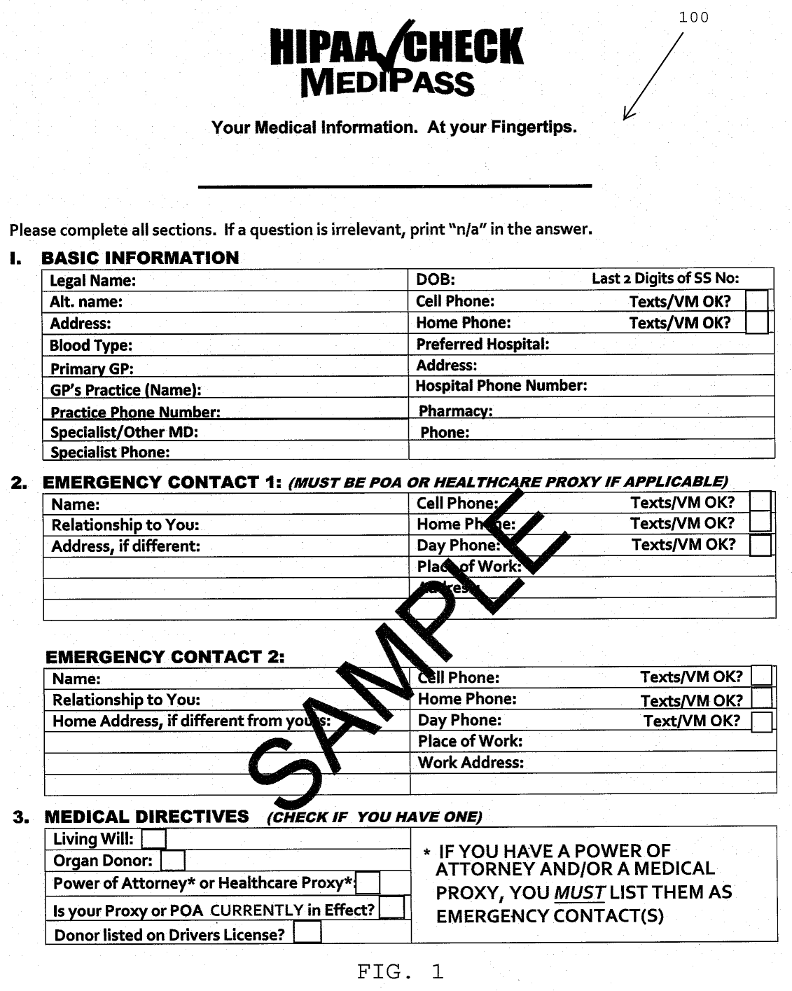

Referring to FIG. 1, in one embodiment, a portable system for accessing medical information preferably includes a form 100 that may be used for collecting medical information for an individual or patient. The form 100 may have different sections for compiling different types of medical information about the individual.

In one embodiment, a first page of the form 100 preferably includes a basic information section, an emergency contact section and a medical directives section. In one embodiment, the basic information section may include but is not limited to the following information: legal name, alternative name, address, blood type, primary physician, the name of the primary physician's practice, primary physician's phone number, specialist/other medical doctor, the specialist's phone number, date of birth, the last two digits of social security number, cell phone number, home phone number, preferred hospital, hospital address, hospital phone number, pharmacy, and pharmacy phone number. In one embodiment, the form 100 preferably includes one or more authorization check boxes authorizing text messages and voice mail messages to be sent to one or more of the telephone numbers that may be listed on the form 100.

In one embodiment, the emergency contact section of the form 100 may include the following information for an emergency contact: the emergency contact's relationship to the individual completing the form, address, cell phone number, home phone number, work phone number, place of work, and work address. In one embodiment, the emergency contact section of the medical information form 100 may include one or more check boxes for authorizing text messages and/or voice mail messages to be sent to one or more of the telephones listed for the emergency contact.

In one embodiment, the emergency contact section of the form 100 may include an area for entering contact information for a second emergency contact, which is generally similar to the type of information that is collected for the first emergency contact.

In one embodiment, the form 100 used for collecting medical information preferably includes a medical directives section whereby an individual or patient may designate and provide information in one or more of the following categories: living will, organ donor, power of attorney, healthcare proxy, an indication if the power of attorney or health care proxy is currently in effect, organ donor listed on driver's license. In one embodiment, the medical directives section of the form 100 requires confirmation that all parties listed as having been granted power of attorney or medical proxy status have also been listed as one of the emergency contacts.

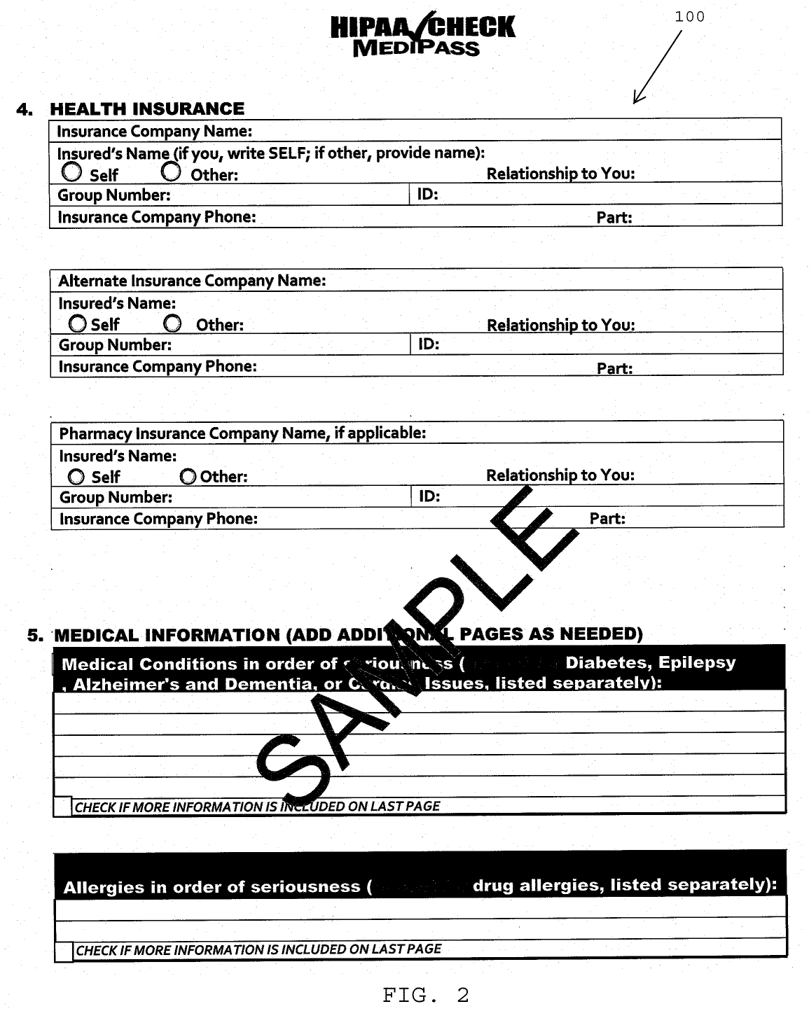

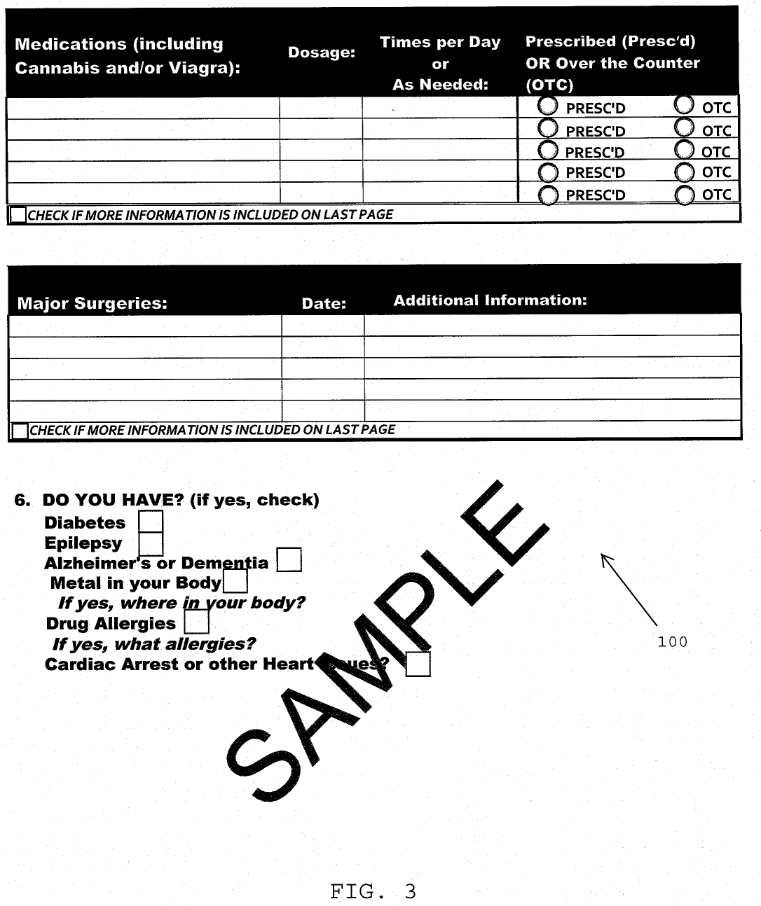

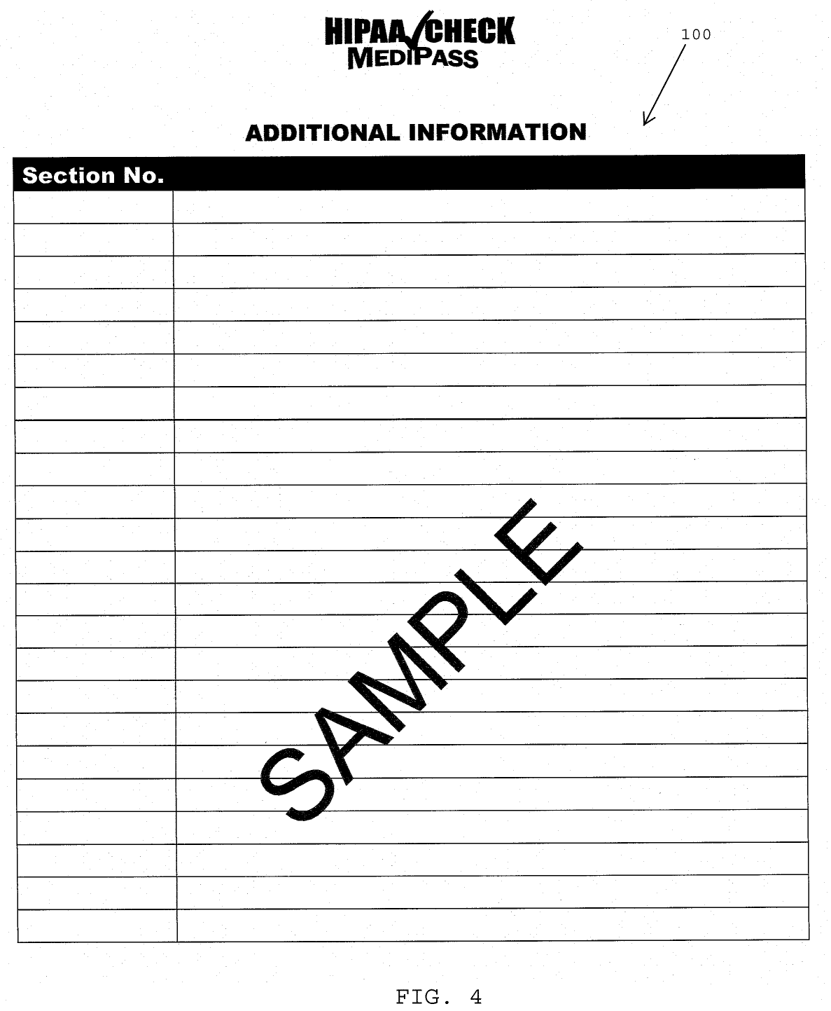

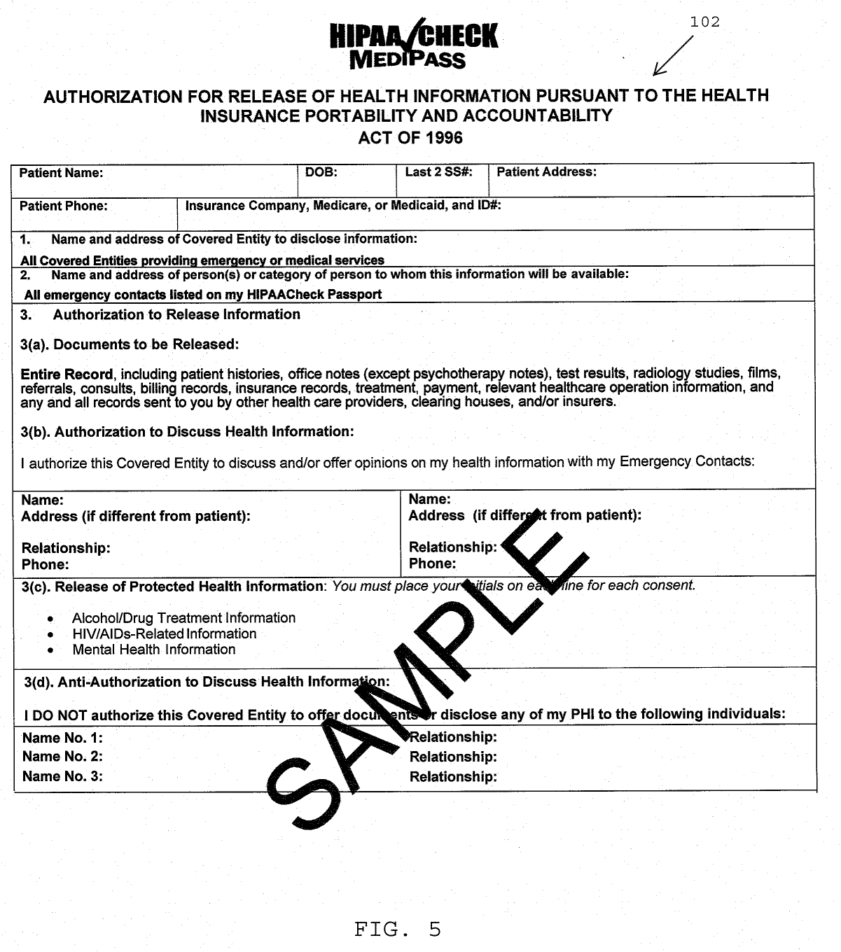

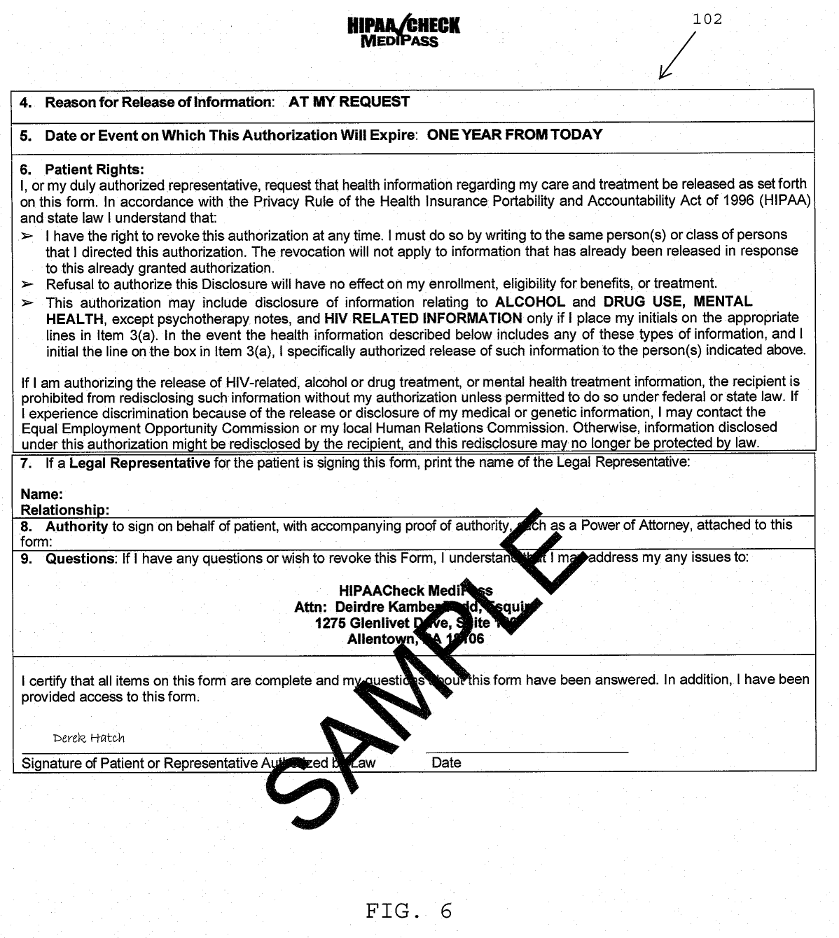

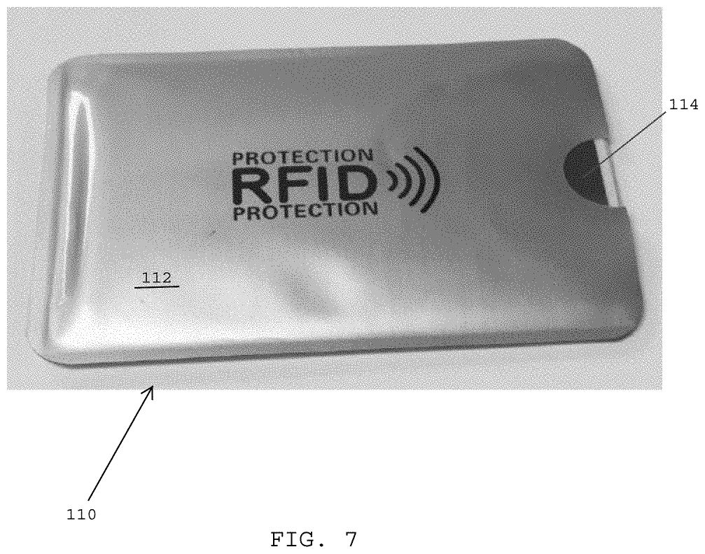

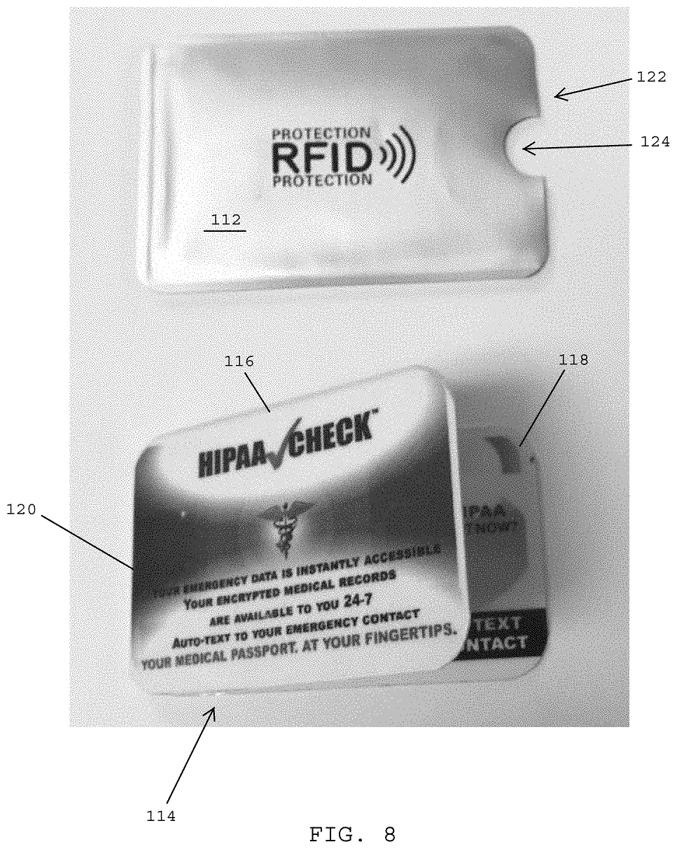

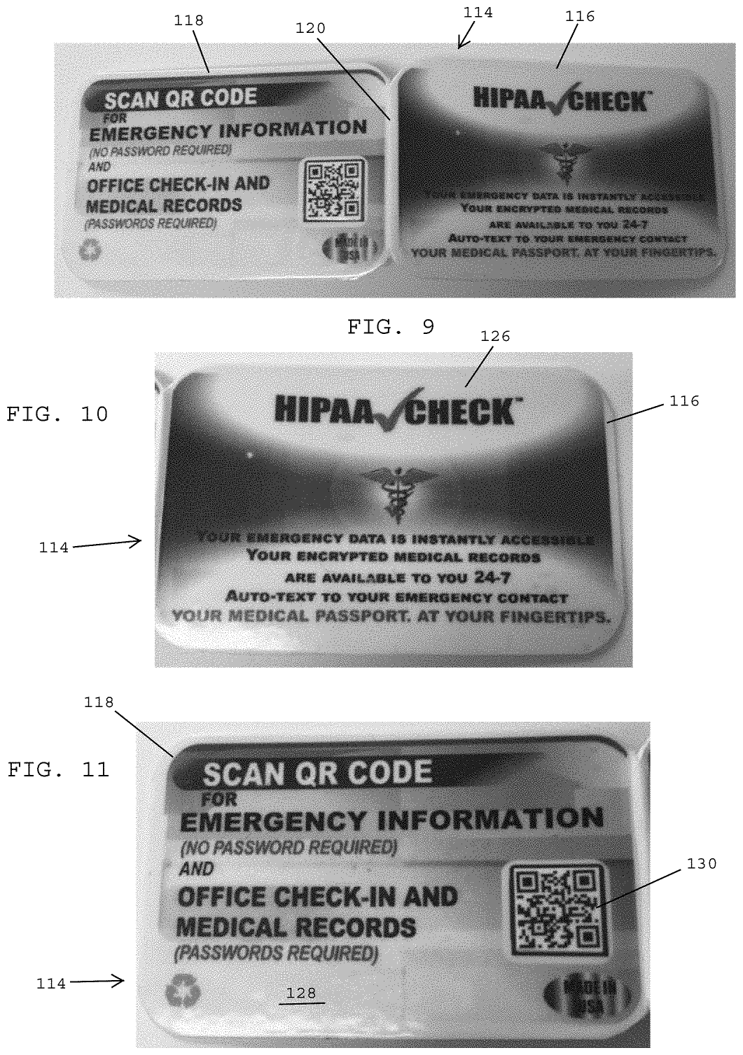

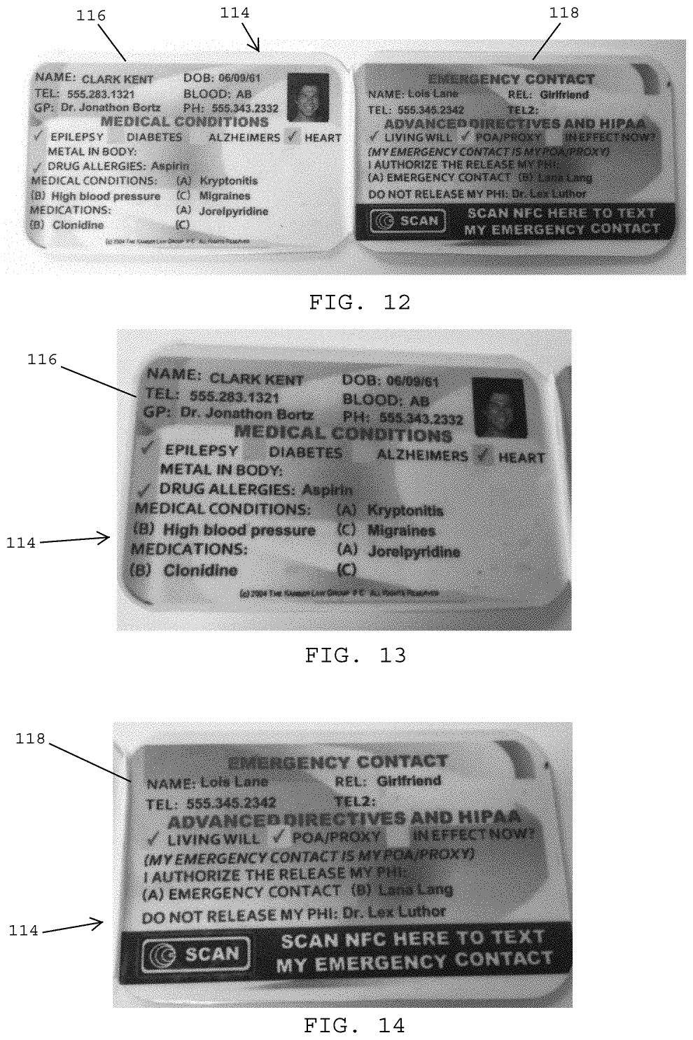

Referring to FIG. 2, in one embodiment, a second page of the form 100 used for collecting medical information preferably includes sections for gathering health insurance information and medical information for the individual or patient completing the form. In one embodiment, the information entered into the health insurance section may include but is not limited to the following: insurance company name, insured's name, group number, and identification number, insurance company phone number.

In one embodiment, the health insurance section may include information for providing an alternate insurance company name, insured's name, group number, identification number, and insurance company phone number.

In one embodiment, the health insurance section of the form 100 used to collect medical information preferably includes pharmacy information such as pharmacy insurance company name, insured's name, group number, identification number, and pharmacy insurance company phone.

In one embodiment, the form 100 used for collecting medical information may include a medical information section in which an individual may list medical conditions. The individual may also identify allergies (e.g., pollen, bees).

Referring to FIG. 3, in one embodiment, the form 100 used for collecting medical information may include a medications section that enables an individual to list and identify any medications and drugs that are being taken such as prescription drugs and over the counter (OTC) medications. The medications section may include an identification of the name or type of the medication, the dosage amount, the number of times per day that the medication is being taken by the individual, and whether the medication is a prescription drug or an over the counter (OTC) medication.

In one embodiment, the medical information section of the form 100 may include an area for listing major surgeries including the type of surgery, the date of the surgery, and any additional information desired to be entered by the individual. In one embodiment, the form 100 for collecting medical information preferably includes a section for indicating major medical conditions (e.g., diabetes, epilepsy, Alzheimer's or dementia, metal in the body, where the metal in the body is located, drug allergies, identifying particular types of allergies, cardiac arrest, and other heart issues).

Referring to FIG. 4, in one embodiment, the form 100 used for collecting medical information preferably includes an additional information section for adding any additional medical information that has not be entered for the first three pages of the form shown in FIGS. 1-3. The additional information section may have rows and/or columns that enable an individual to refer back to one of the previous sections of the form 100 for adding the additional information.

For the form 100 shown and described above in FIGS. 1-4, it is contemplated that any type of information that is required by doctors, medical personnel, medical offices, surgical centers, hospitals, pharmacies, insurance companies, governments, schools and regulators for treating individuals and patients may be added to the form. The information that is collected on the form 100 is preferably added to a computer database that is linked to a particular individual or patient, so that the medical information may be accessed via the internet and websites when needed for treating or assisting the particular individual or patient.

Referring to FIGS. 5 and 6, in one embodiment, a system for providing medical information preferably includes a health information release form 102 for authorizing the release of health information pursuant to the Health Insurance Portability and Accountability Act of 1996 (HIPAA). The health information release form 102, hereinafter referred to as the HIPAA form 102, preferably includes sections designating a patient, and organizations and individuals that may be authorized to receive health care and medical information about the designated individual or patient. The HIPAA form 102 may include an anti-authorization section, which provides information about individuals that are not authorized to receive health and medical information about the patient that is designated on the HIPAA form 102.

Referring to FIG. 6, in one embodiment, the HIPAA form 102 preferably identifies the patient's rights and provides information regarding legal representative and power of attorney forms that may be attached to the HIPAA form 102. The HIPAA form 102 preferably includes signature and date blocks to be completed by the individual or patient, or by a representative authorized by law to sign on behalf of the individual/patient.

The information that is gathered by using the form 100 for collecting medical information of FIGS. 1-4 and the HIPAA form 102 of FIGS. 5 and 6 is preferably entered into one or more computer databases that are designed to protect the privacy of the information for the patient and limit access to the information to only those personnel who are authorized to access the information. When necessary, the medical information and the HIPAA authorization information compiled in the one or more computer databases may be accessed for treating a patient, or for facilitating the medical treatment of the patient. In one embodiment, the information may be cataloged into publicly available information, which is available at a lower security level, and more private medical information that requires a higher level of security to attain access. In one embodiment, a password may be required to access certain types of medical information.

Referring to FIG. 7, in one embodiment, a system for accessing medical information preferably includes a protective sleeve 112 that is adapted to hold a medical information passport 114. In one embodiment, the medical information passport 114 is insertable into the protective sleeve 112 for storage, and is removable from the protective sleeve for use. In one embodiment, the medical information passport 114 may contain the medical information and/or the HIPAA information that is gathered using the forms 100 and 102 shown and described above in FIGS. 1-6, or provides a link for accessing the medical information and/or the HIPAA information via the internet or a Cloud computing system.

As used herein, the terminology “Cloud computing” means the on-demand availability of computer system resources, especially data storage and computing power, without direct active management by the user. The term is generally used to describe data centers available to many users over the Internet. Large Cloud systems often have functions distributed over multiple locations from central servers. Clouds may be limited to a single organization (i.e., enterprise clouds), may be available to many organizations (i.e., public clouds), or a combination of both (i.e., hybrid clouds). One of the largest public clouds is known by the name Amazon AWS.

In one embodiment, the medical information passport 114 desirably has a near field communication (NFC) tag incorporated therein, and the protective sleeve 112 preferably includes a material (e.g., metal, aluminum) that prevents unauthorized access to the NFC tag, the information on the NFC tag, and/or the information contained on or linked to the medical information passport 114.

As used herein, the terminology near field communication (NFC) means a set of communication protocols that enable two electronic devices, one of which is usually a portable device such as a smart phone, to establish communication by bringing the two devices close to one another (e.g., within 1.6 inches of one another). NFC tags are passive data stores which can be read, and under some circumstances written to, by an NFC device. NFC tags typically contain data and are read-only in normal use, but may be rewritable. Applications include secure personal data storage (e.g. credit card information, and personal identification numbers). NEC tags can be custom-encoded by their manufacturers or use industry established specifications.

Peer-to-peer communication is a feature that sets NFC technology apart from typical Radio-frequency identification (RFID) devices. An NFC device is able to act as both a reader and as a tag. This unique ability has made NFC technology a popular choice for contactless data transmission, a key driver in the decision by influential players in the mobile phone industry to include NFC technology in newer smart phones. In addition, NFC-equipped smart phones are able to pass along information from one smart phone to another by tapping the two devices together, which turns data sharing into a simple task.

A criminal can take advantage of NFC technology by using a scanner that wirelessly scans a victim's NFC device in the same way that a cash register scans it. A criminal can hide the scanner inside a glove or a bag, and then place himself close to the victim and wirelessly steal the victim's information.

In one embodiment, is order to prevent NFC skimming, the protective sleeve is made of a material, such as aluminum, that interferes with radio waves. The protective sleeve is preferably constructed to function as a Faraday cage, which blocks all electromagnetic fields and prevents communication between an illegal scanning device and the NFC tag.

FIG. 8 shows the medical information passport 114 after it has been removed from the protective sleeve 112, whereupon the NFC tag may be scanned. The medical information passport 114 preferably includes a first half 116 that is folded over a second half 118 along a fold line 120. When the medical information passport 114 is in a folded configuration, with the first half 116 overlying the second half 118, the medical information passport 114 is preferably insertable into an open end 122 of the protective sleeve 112. The open end 122 of the protective sleeve desirably includes a cutout 124 for accessing an outer edge of the medical information passport 114 after it has been inserted into the protective sleeve 112 (FIG. 7).

Referring to FIG. 9, in one embodiment, the medical information passport 114 desirably includes the first and second halves 116, 118 that are connected to one another along the fold line 120. The medical information passport 114 may be made of paper, such as recycled paper, that is coated with a clear film, which enables information that is printed on the paper to be read through the clear coating.

Referring to FIGS. 9 and 10, the first half 116 of the medical information passport preferably has an outer surface 126 that contains printed information such as some of the capabilities of the medical information passport.

Referring to FIGS. 9 and 11, in one embodiment, the second half 118 of the medical information passport 114 preferably has an outer surface 128 that has a QR code 130 printed thereon, which may be scanned. Scanning the QR code may provide a link to one or more computer databases where medical information about the individual carrying the medical information passport may be obtained.

As used herein, a OR code (abbreviated from Quick Response Code) means a type of two-dimensional barcode, which was first designed in 1994 in Japan for the automotive industry. A barcode is a machine-readable optical label that contains information about the item to which it is attached. In practice, QR codes often contain data that points to a website or application.

The OR system became popular outside the automotive industry due to its fast readability and greater storage capacity compared to standard barcodes. Applications include product tracking, item identification, time tracking, document management, and general marketing.

A QR code comprises black squares arranged in a square grid on a white background, which can be read by an imaging device such as a camera, and processed using software until the image is appropriately interpreted. The required data is then extracted from patterns that are present in both horizontal and vertical components of the image.

In one embodiment, scanning the QR code 130 provides emergency information about the individual carrying the medical information passport 114, such as major medical conditions, allergies, etc. In one embodiment, scanning the OR code 130 will enable the individual carrying the medical information passport to complete an office check-in and enable medical personnel to obtain medical records, insurance information, pharmacy information, etc. for the individual/patient. In one embodiment, obtaining access to the office check-in information and/or the medical records information may require using one or more passwords.

In one embodiment, all of the information that may be obtained by scanning the OR code is preferably stored in one or more computer databases that may be accessed through the internet and/or a Cloud-based storage system.

Referring to FIG. 12, in one embodiment, the medical information passport 114 preferably includes inner faces of the first and second halves 116, 118 (FIG. 9). Referring to FIGS. 12 and 13, in one embodiment, the first half 116 of the medical information passport 114 preferably has an inner face 132 that includes printed information about the individual carrying the medical information passport 114. In one embodiment, the inner face 132 may include the following information for an individual/patient printed thereon: name, date of birth, blood type, telephone number, name of treating physician, phone number of treating physician and a picture of the individual. In one embodiment, the inner face 132 of the first half 116 of the medical information passport 114 may include information about medical conditions of the individual/patient (e.g., epilepsy, diabetes, Alzheimer's, heart ailments, whether metal is in the body, drug allergies, and medications taken by the individual.

Referring to FIGS. 12 and 14, in one embodiment, the medical information passport 114 preferably includes the second half 118 having an inner face 134 that has printed thereon emergency contact information including but not limited to the name of the emergency contact, the relationship of the emergency contact to the patient, and one or more telephone numbers for the emergency contact.

In one embodiment, the inner face 134 of the second half 118 may also include advanced directives and HIPAA release information such as whether the individual carrying the medical information passport 114 has a living will, a power of attorney, a proxy, and whether any of the living will, power of attorney, and proxy are in effect. The inner face 134 of the second half 118 may also include information linking the emergency contact with the power of attorney and proxy, and authorizing the release of personal health information to an emergency contact. The inner face 134 may also include printed information that identifies an individual that is not authorized to receive personal health information about the patient. The inner face 134 may also include an indication that the second half 118 of the medical information passport 114 has near field communication (NFC) technology incorporated therein, whereby an emergency medical technician or first responder may scan an NFC tag to generate a text message that will be automatically transmitted to the emergency contact to notify the emergency contact that the individual carrying the medical information passport is undergoing emergency medical treatment. In one embodiment, when the NEC tag is scanned or detected by a sensor/cell phone, the sensor/cell phone will automatically send a text message to the cell phone of the emergency contact.

Referring to FIG. 15, in one embodiment, a portable system for providing medical information may include a bracelet 140 that is worn about an individual's/patient's wrist. The bracelet may be in addition to the medical information card that is carried by the individual. In one embodiment, the bracelet 140 may include a section 142 (e.g., a band) that provides an alert or indication that the bracelet is being utilized as a medical bracelet that contains medical information about the wearer. In one embodiment, the bracelet 140 preferably includes a near field communication (NFC) tag 144 secured thereto, which may be scanned using a cell phone or an NEC tag sensor to automatically generate a text message of a voice message that is transmitted to a pre-determined emergency contact. The text message will preferably notify the emergency contact that the individual wearing the bracelet 140 is undergoing emergency medical treatment. The text message may also include information regarding the location of the patient, the organization that is treating the patient, and how the emergency contact may reach the organization that is treating the patient (e.g., a phone number for the hospital that is treating the patient).

Referring to FIG. 16, in one embodiment, in addition to wearing the bracelet shown in FIG. 15, an individual or patient may wear a watch having an NFC tag or technology incorporated therein. The watch 146 is preferably worn about the wrist of a user and first responders may scan the watch to automatically generate a text message or an emergency call to an emergency contact that has been linked with the watch 146. In one embodiment, the front face of the watch 146 may include an indication (e.g., a symbol or a message printed on the front face) that the device is being used as a medical alert device to notify medical personnel and individuals treating a patient that the watch may be used to send text messages or voice mails to emergency contacts.

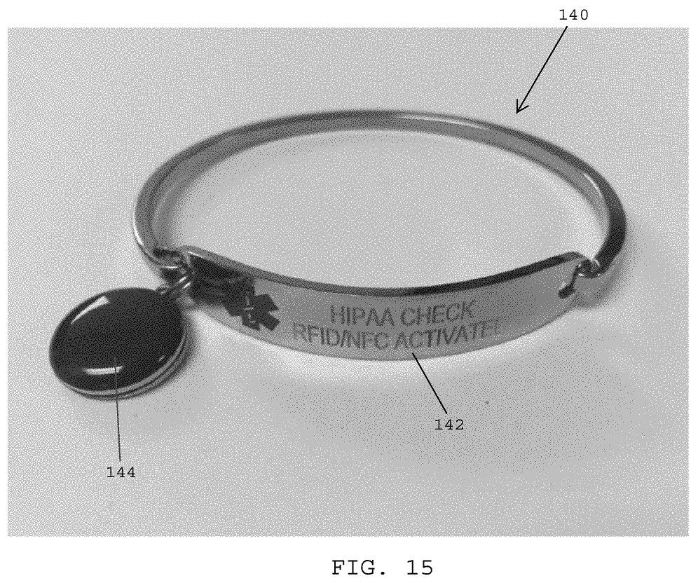

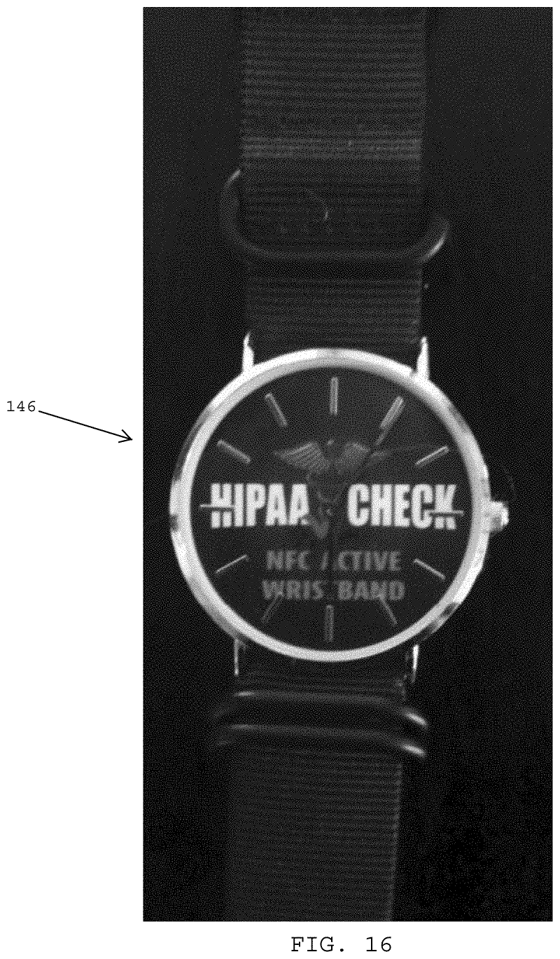

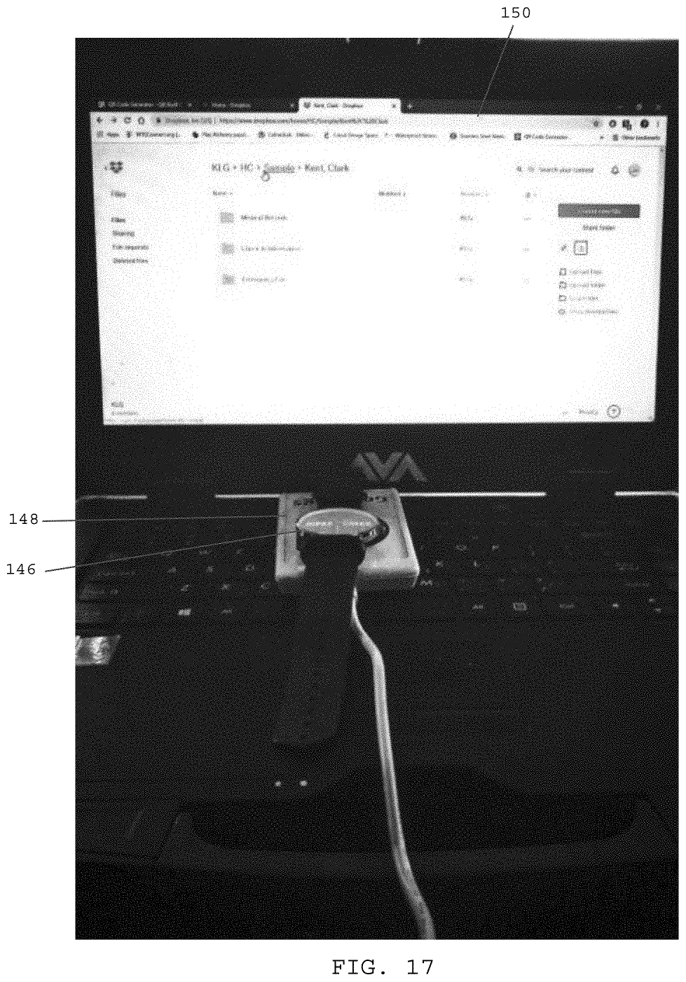

Referring to FIG. 17, in one embodiment, the watch 146 may be programmed by a user by placing the watch 146 upon a programming platform 148 that is linked with a computer system 150. After communication has been established between the watch 146 and the computer system 150, a programmer or a user of the watch 146 may enter information into the computer database to provide emergency contact information. The emergency contact information entered into the watch may then be utilized to send text messages to one or more emergency contacts. In lieu of text messages, the watch 146 may be programmed to generate a telephone call to a cell phone or smart phone of one or more emergency contacts.

Referring to FIG. 18, in one embodiment, a portable system for accessing medical information may be used for collecting medical information for a dependent, such as a child or a dependent adult (e.g., dementia patient) who is not independent. In one embodiment, the system preferably includes a dependent medical information form 152 for collecting medical information about a dependent. In one embodiment, the information submitted using the dependent medical information form 152 is preferably in addition to the information submitted using the form 100 (FIGS. 1-4) and the HIPAA form 102 (FIGS. 5-6) described herein. In one embodiment, the dependent medical information form 152 may be used to collect various types of information about a child or a dependent including but not limited to the following: name, nickname, address, height, age, weight, name of caretaker, parent or guardian address, shoe size, hair color, eye color, ethnicity, primary languages, medical conditions, daily medications, mental limitations, physical limitations, marks, scars, piercings, tattoos, eye glass status, teeth braces status, and allergies.

In one embodiment, the dependent medical information form 152 may include a first image showing the front side of a body, and a second image showing the back side of a body. The body images may be marked up and/or annotated to show the location of marks, scars, piercings, or tattoos that appear on a body, including a description of the appearance of the marks, scars, piercings, or tattoos (e.g., a butterfly tattoo).

In one embodiment, the dependent medical information form 152 for collecting medical information for a child or a dependent preferably includes areas for collecting fingerprints for the left and the right hands of the child or the dependent including the left and right pinky fingers, the left and right ring fingers, the left and right middle fingers, the left and right index fingers, and the left and right thumbs.

In one embodiment, the information collected using the dependent medical information form 152 is preferably entered into one or more computer databases so that the information may be accessed at a later time using the NFC devices, OR codes, USB flash drives, and/or portable memory devices disclosed herein.

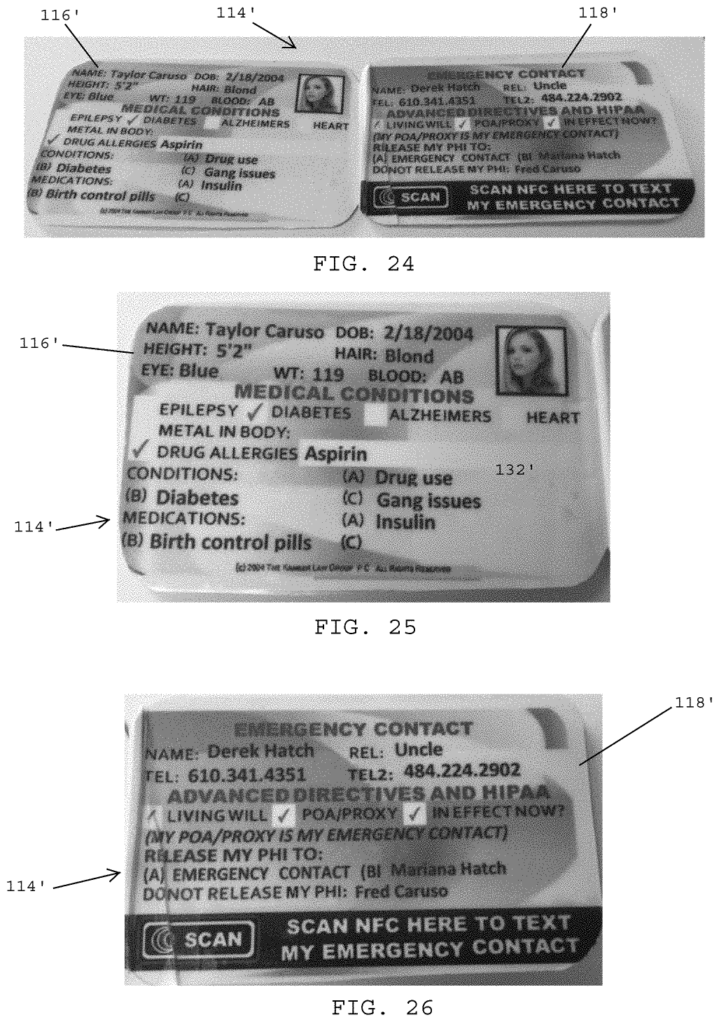

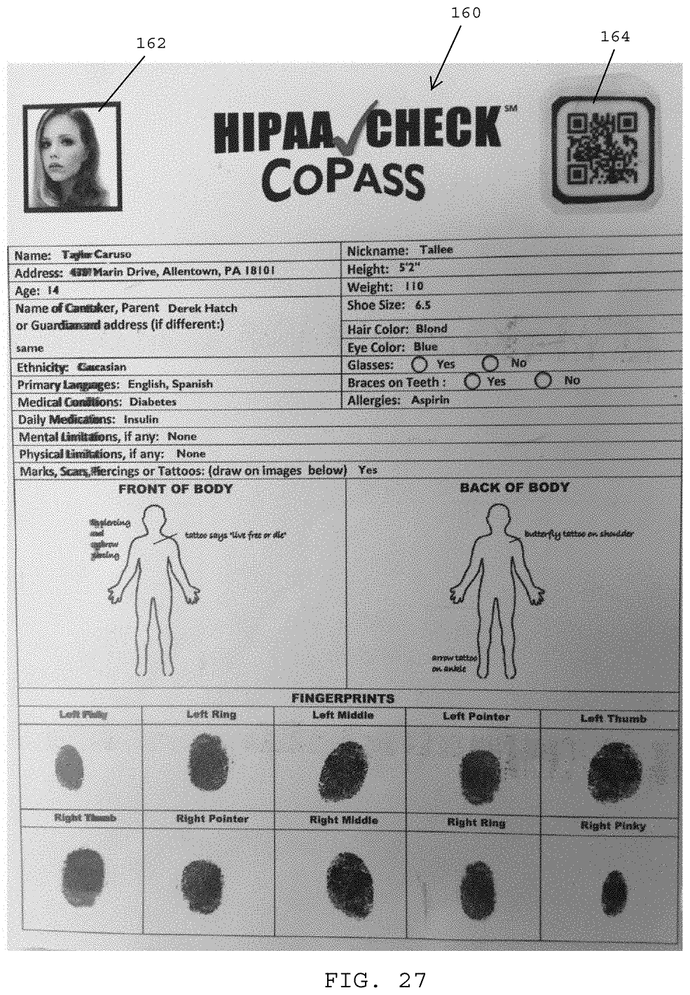

Referring to FIG. 19, in one embodiment, a system for collecting medical and identifying information to be added to the dependent medical form 152 of FIG. 18 may include cotton swabs 154 that are utilized for obtaining DNA samples. For example, the cotton swabs 154 may be utilized for obtaining a saliva sample for an individual. In one embodiment, the system may include an ink pad 156 that provides ink for fingertips for obtaining fingerprints on the left and right hands of an individual. In one embodiment, the system may include a storage pouch 158 for submitting the collected samples, such as DNA and fingerprint samples, for inclusion on the display sheet 160 (FIG. 27).

Referring to FIG. 20, in one embodiment, a portable system for accessing medical information preferably includes a protective sleeve 112′ and a medical information passport 114′ that is inserted into the protective sleeve 112′ for storage and when not in use. In one embodiment, the medical information passport 114′ has near field communication (NFC) technology incorporated therein (e.g., an NFC tag), and the protective sleeve 112′ includes a material (e.g., metal) that prevents unauthorized access of the information that is contained on the medical information passport 114′. In one embodiment, the protective sleeve 112′ is about the size of a credit card and may be carried in a wallet.

FIG. 20 shows the medical information passport 114′ after it has been removed from the protective sleeve 112′. The medical information passport 114′ preferably includes a first half 116′ that is folded over onto a second half 118′ along a fold line 120′. When the medical information passport 114′ is in a folded configuration with the first half 116′ overlying the second half 118′, the medical information passport 114′ is preferably insertable into an open end 122′ of the protective sleeve 112′. The open end 122′ of the protective sleeve desirably includes a cutout 124′ for accessing an edge of the medical information passport 114 after it has been inserted into the protective sleeve 112′.

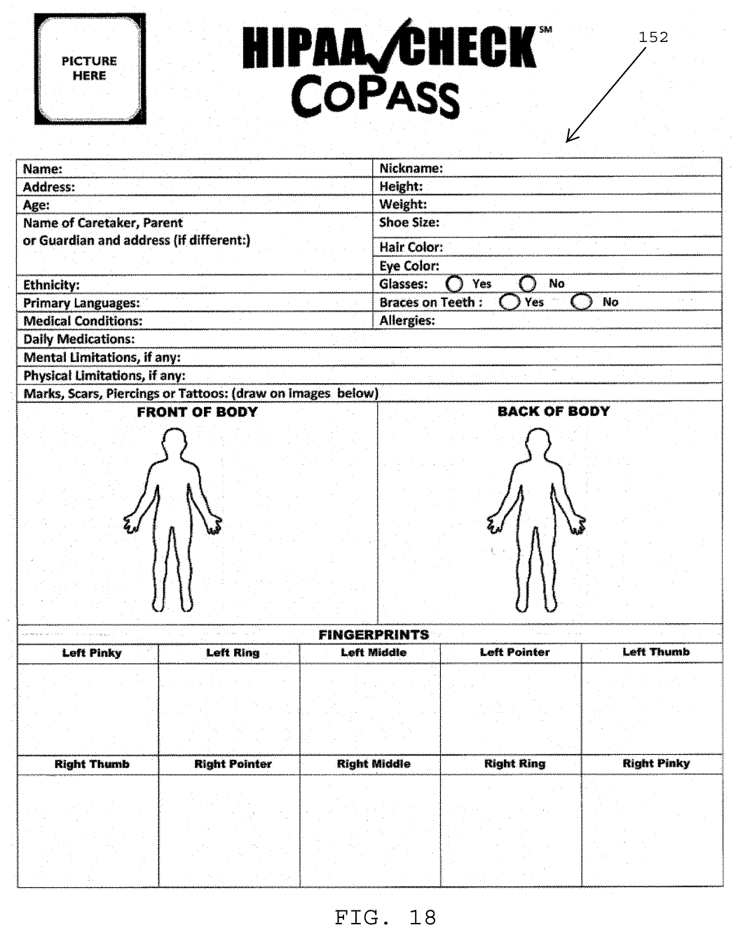

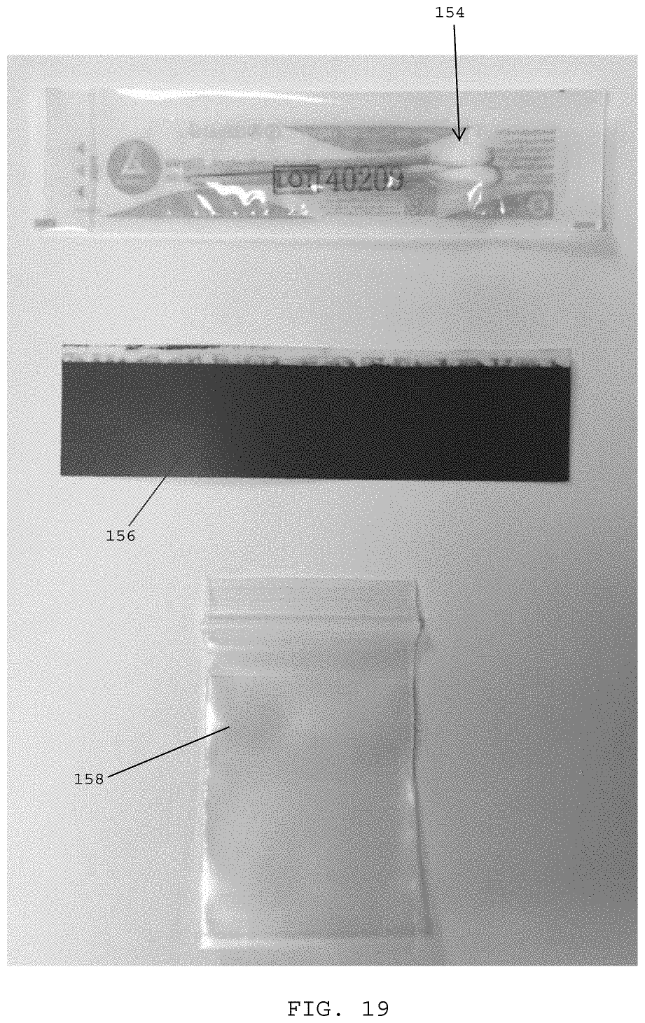

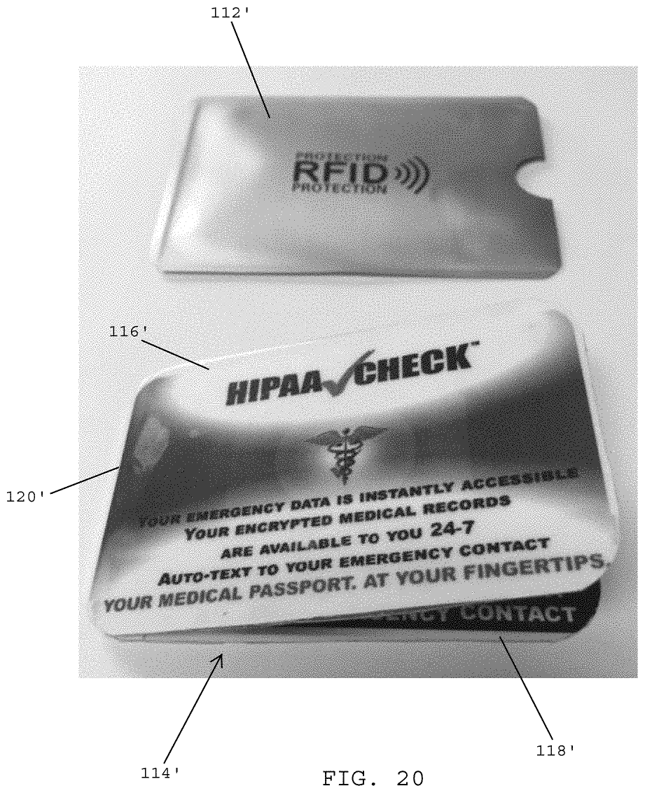

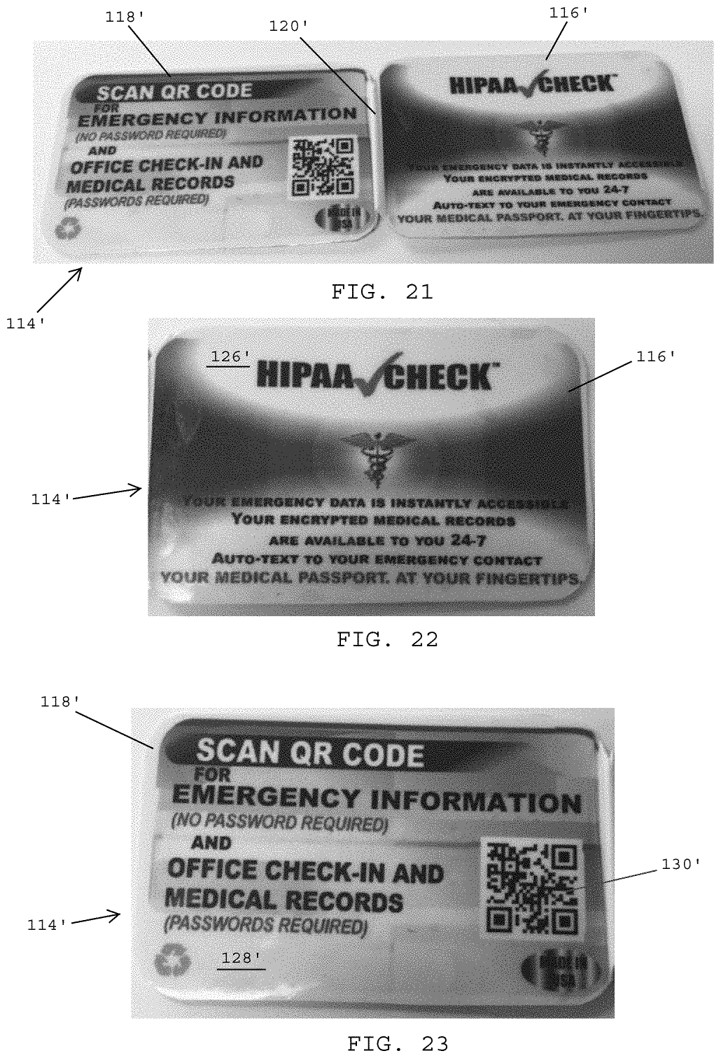

Referring to FIG. 21, in one embodiment, the medical information passport 114′ desirably includes the first half 116′ and the second half 118′ that are connected to one another along the fold line 120′. The medical information passport 114′ may be made of paper, such as recycled paper, that is coated with a clear film, which enables information that is printed on the paper to be read through the clear coating.

Referring to FIGS. 21 and 22, the first half 116′ of the medical information passport 114′ preferably has an outer surface 126′ that contains printed information such as some of the capabilities of the portable system that incorporates the medical information passport.

Referring to FIGS. 21 and 23, in one embodiment, the second half 118′ of the medical information passport 114′ preferably has an outer surface 128′ that has a QR code 130′ printed thereon, which may be scanned, Scanning the QR code may provide a link to one or more computer databases where medical information about the individual carrying the medical information passport may be obtained. In one embodiment, scanning the QR code 130′ provides emergency information about the individual carrying the medical information passport 114′, such as major medical conditions, allergies, etc. In one embodiment, the emergency information may be obtained without using a password. In one embodiment, scanning the OR code 130′ will enable the individual carrying the medical information passport to complete an office check-in and/or enable medical personnel to obtain medical records, insurance information, pharmacy information, etc. for the individual/patient. In one embodiment, obtaining access to the office check-in information and/or the medical records information requires using a password.

In one embodiment, all of the information that may be obtained by scanning the QR code is preferably stored in one or more computer databases that may be accessed through the internet and/or a Cloud-based storage system.

Referring to FIG. 24, in one embodiment, the medical information passport 114′ preferably includes inner faces of the first and second halves 116′, 118′ (FIG. 21). Referring to FIGS. 24 and 25, in one embodiment, the first half 116′ of the medical information passport 114′ preferably has an inner face 132′ that includes printed information about the individual carrying the medical information passport 114′. In one embodiment, the inner face 132′ may include the following information for an individual/patient printed thereon: name, date of birth, blood type, telephone number, name of treating physician, phone number of treating physician and a picture of the individual. In one embodiment, the inner face 132′ of the first half 116′ of the medical information passport 114′ may include information about medical conditions of the individual/patient (e.g., epilepsy, diabetes, Alzheimer's, heart ailments, whether metal is in the body, drug allergies, and medications taken by the individual.

Referring to FIGS. 24 and 26, in one embodiment, the medical information passport 114′ preferably includes the second half 118′ having an inner face 134′ that has printed thereon emergency contact information including but not limited to the name of the emergency contact, the relationship of the emergency contact to the patient, and one or more telephone numbers for the emergency contact.

In one embodiment, the inner face 134′ of the second half 118′ may also include advanced directives and HIPAA release information such as whether the individual carrying the medical information passport 114′ has a living will, a power of attorney, a proxy, and whether any of the living will, power of attorney, and proxy are in effect. The inner face 134′ of the second half 118′ may also include information linking the emergency contact with the power of attorney and proxy, and authorizing the release of personal health information to an emergency contact. The inner face 134′ may also include printed information that identifies an individual that is not authorized to receive personal health information about the patient. The inner face 134′ may also include an indication that the second half 118′ of the medical information passport 114′ has near field communication (NFC) technology incorporated therein, whereby an emergency medical technician or first responder may scan an NFC tag to generate a text message that will be automatically transmitted to the emergency contact to notify the emergency contact that the individual carrying the medical information passport is undergoing emergency medical treatment. In one embodiment, when the NFC tag is scanned or detected by a sensor/cell phone, the sensor/cell phone will automatically send a text message to the cell phone of the emergency contact. Voice mail messages may also be generated and transmitted.

Referring to FIG. 27, in one embodiment, the medical information collected using the dependent form 152 shown in FIG. 18 may be printed on a dependent medical information sheet 160 that displays medical and identifying information about a child or a dependent adult. In one embodiment, the dependent medical information sheet 160 may have a magnetic backing that enables the sheet 160 to be placed on an object having a metallic surface such as a refrigerator or a filing cabinet. In one embodiment, the display sheet 160 preferably includes a picture area 162 that has a picture of a child or a dependent affixed thereto or printed thereon. The dependent information sheet 160 may include a QR code 164 that may be scanned to link to one or more computer databases that contain the medical information for the child or dependent adult. The dependent medical information sheet 160 may include the information collected using the dependent form 152 shown and described above in FIG. 18. The dependent medical information sheet 160 may include a first image of a front side of a body and a second image of a back side of a body to show all of the marks, scars, piercings, or tattoos that appear on a child or a dependent adult, as well as the location of the marks, scars, piercings, or tattoos. The dependent medical information sheet 160 may also include comments on the type of mark, scar, piercings or tattoos such as “butterfly tattoo on shoulder” or “tattoo that says ‘live free or die’ on the chest.”

In one embodiment, the dependent medical information sheet 160 may include fingerprints for each of the five fingers on the left and right hands of a child or a dependent adult.

Referring to FIG. 28, in one embodiment, the dependent medical information sheet 160 shown and described above in FIG. 27 may have a magnetic backing for being secured to a metallic surface such a surface of a front door of a refrigerator 168. In one embodiment, a magnetic backing on the dependent medical information sheet 160 is attracted to the metallic surface of the front door of the refrigerator 168. The dependent medical information sheet 160 having a magnetic backing may also be secured to any metallic surface such as a surface of a filing cabinet.

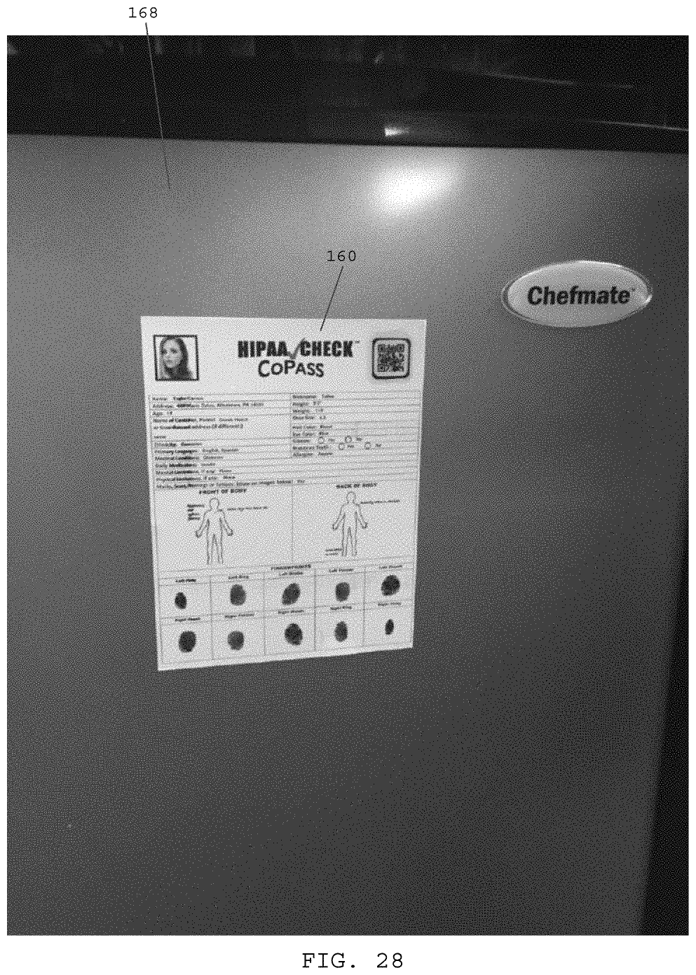

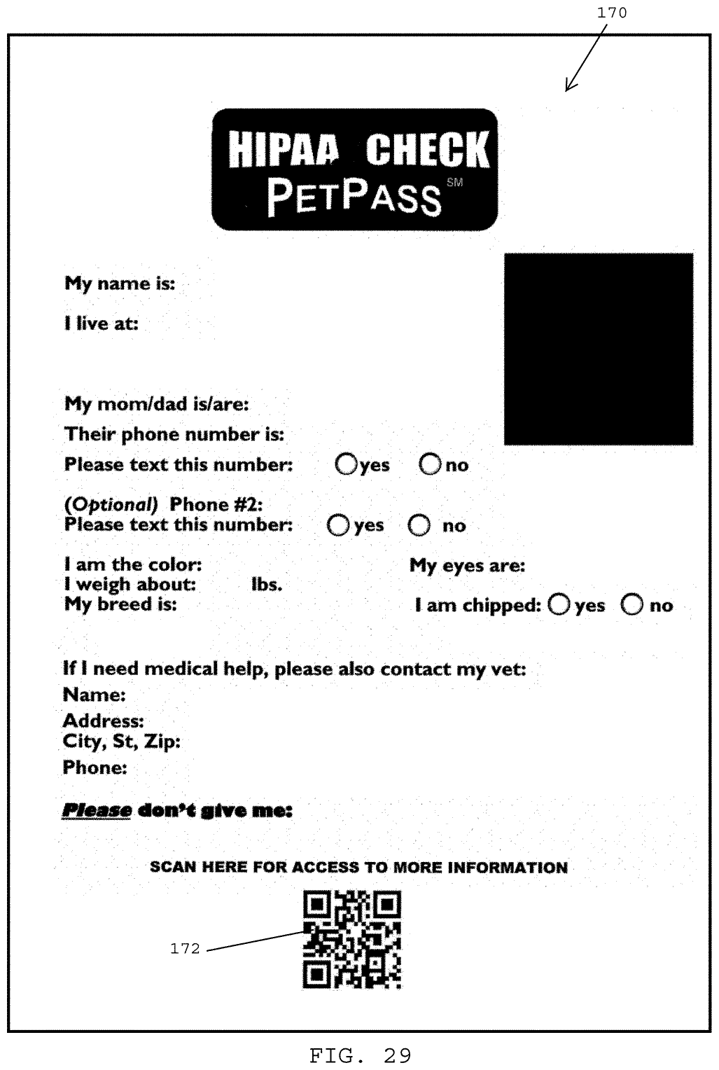

Referring to FIG. 29, in one embodiment, a pet medical information form 170 may be utilized for collecting medical information about a pet. In one embodiment, the pet medical information form 170 may be used to collect information about a pet including but not limited to the following: the name of the pet, the address of the pet, the parents of the pet, the phone number of the parents, whether a text may be sent to the phone number, an alternate phone number, the color of the pet, the weight of the pet, the breed of the pet, the eye color of the pet, and whether the animal has an embedded identification chip (e.g., an RFID chip). In one embodiment, the pet medical information form 170 may also be used to collect information about medical personnel including but not limited to the following: the name of the pet's veterinarian, the address of the veterinarian, and the phone number of the veterinarian. The pet medical information form 170 may include an area for identifying foods or drugs that should not be given to the pet. In one embodiment, the pet medical information form 170 preferably includes a QR code that may be scanned to access one or more computer databases that contain the identifying and/or medical information associated with the pet. The OR code 172 may also be provided on an item secured to the pet such as a tag secured to a pet collar.

Referring to FIGS. 30A and 30B, in one embodiment, a pet medical information passport 200 preferably includes identifying and medical information associated with a pet, which may have been collected using the pet medical information form shown and described herein. In one embodiment, the pet medical information passport 200 preferably includes a USB flash drive 202 having USB connectors that enable the USB flash drive 202 to be connected with a communication port of a computer for transmitting medical information and/or medical records to the computer.

As used herein, a USB flash drive, also known as a thumb drive or a memory stick, means a data storage device that includes flash memory with an integrated USB interface. USB flash drives are typically removable, rewritable and much smaller than optical discs. USB flash drives are often used for storage, data back-up and transfer of computer files. Compared with floppy disks or compact discs (CDs), they are smaller, faster, have significantly more capacity, and are more durable due to a lack of moving parts. USB flash drives may have storage capacities of 8-256 GB or more.

The USB flash drive 202 is preferably coupled with the pet medical information card 200 via a swivel connection 204 that enables the USB flash drive 202 to rotate from a retracted configuration shown in FIGS. 30A and 30B to an extended configuration, which will be described in more detail herein. FIG. 30A shows a front face of the pet medical information passport 200 and FIG. 30B shows a rear face of the pet medical information passport 200.

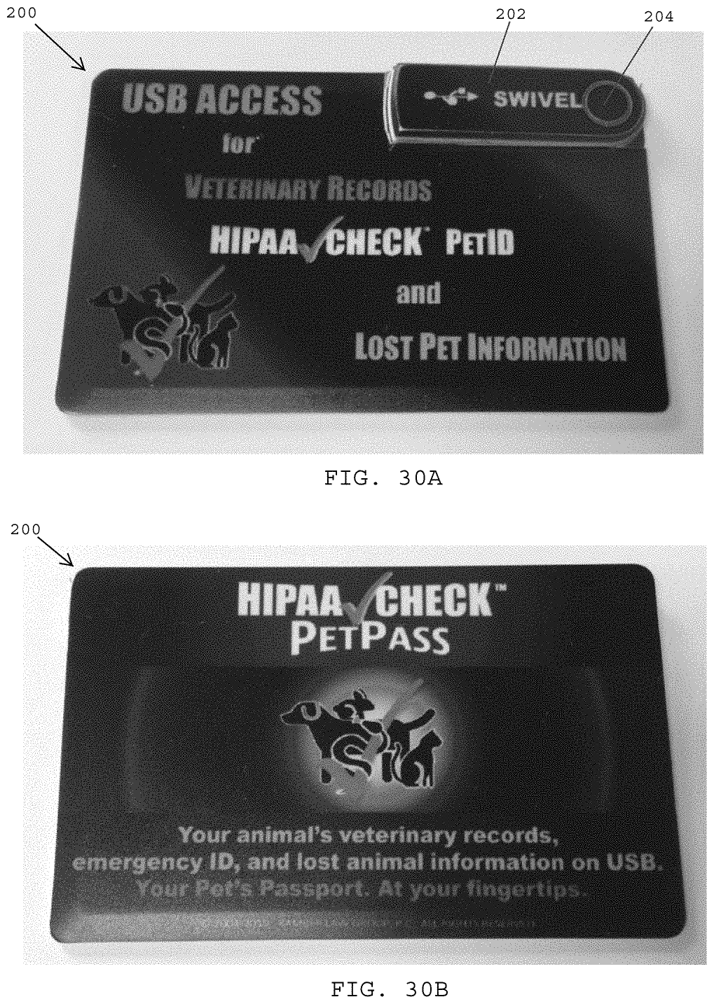

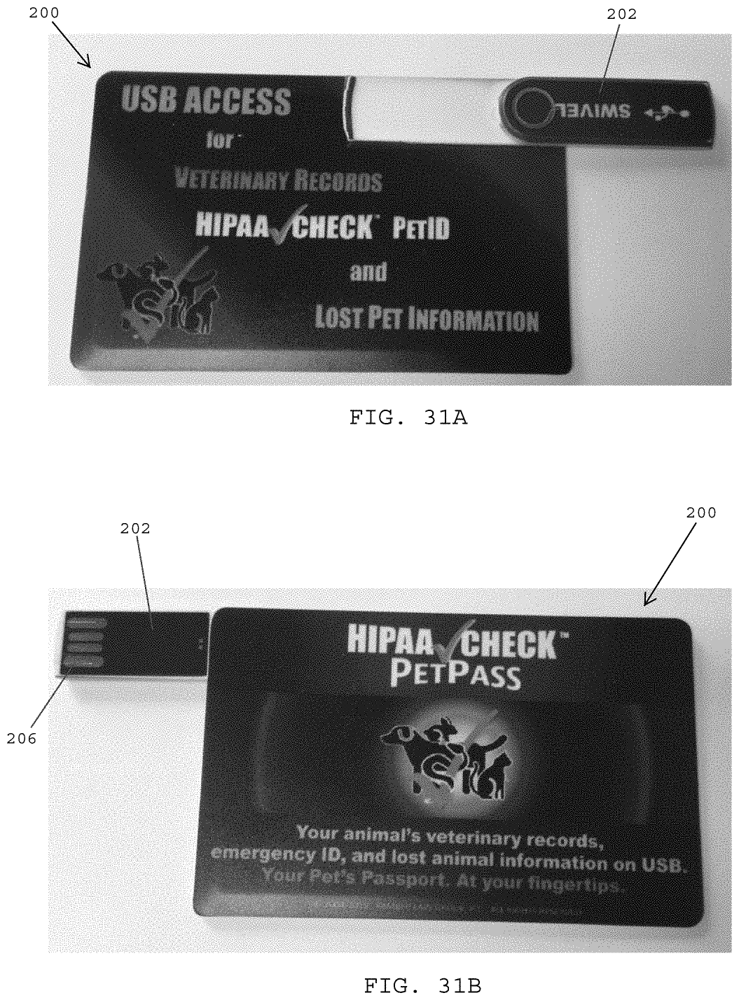

Referring to FIGS. 31A and 31B, the USB flash drive 202 may be rotated or swung to the extended position for exposing the USB connectors 206. When the USB connectors 206 are inserted into a USB port of a computer, the USB flash drive 202 preferably provides information regarding an animal's veterinary records, emergency identification information, and lost animal information. In one embodiment, the pet medical information passport 200 may have a OR code that may be scanned for obtaining access to medical information associated with the pet. In one embodiment, the pet medical information passport 200 may have an NFC tag that may be scanned to provide access to medical information about a pet or may automatically transmit a message to an emergency contact, such as a pet owner.

Referring to FIGS. 32A and 32B, in one embodiment, a portable system for providing medical information for a pet preferably includes a pet tag 210 that may be utilized in conjunction with the pet medical information passport 200 shown and described above in FIGS. 30A-31B. Referring to FIG. 32A, in one embodiment, the pet tag 210 preferably includes a loop 212 for securing the pet tag 210 to an animal collar. The pet tag 210 preferably includes a front face 214 that shows a pet's name 216 (e.g., Whiskey). The front face 214 of the pet tag 210 may also provide information that the pet tag has a OR code and/or an NFC device incorporated therein for identifying and contacting an owner or providing medical and insurance information for the pet. In the particular embodiment shown in FIG. 32A, the front face 214 indicates that the OR code may be scanned for accessing medical information associated with the pet, and that the NFC device may be scanned to message an owner (e.g., text message, VM message, phone call) that the pet wearing the pet tag 210 is lost, injured, or undergoing medical treatment.

Referring to FIG. 32B, in one embodiment, the pet tag 210 preferably has a rear face 218 having a OR code 220 that may be scanned to provide identifying and medical information about the pet wearing the pet tag 210. In one embodiment, information entered into a computer database using the pet medical information form 170 of FIG. 29 will be linked with the OR code 220 provided on the pet tag 210. Moreover, the OR code 220 provided on the pet tag 210 of FIG. 32B may be associated and/or linked with the OR code 172 appearing on the form 170 shown in FIG. 29.

Referring to FIG. 33A and 33B, in one embodiment, a system for notifying an owner that a pet has been lost may include a pet tag 210′ having an NFC device incorporated therein. In one embodiment, the pet tag 210′ has a front face 214′ with printed information regarding the name 216′ of the pet and to notify third parties that the pet tag 210′ may be scanned using a smart phone or configured electronic device to automatically generate a text message to a pet owner at a telephone number that has been pre-programmed into a system.

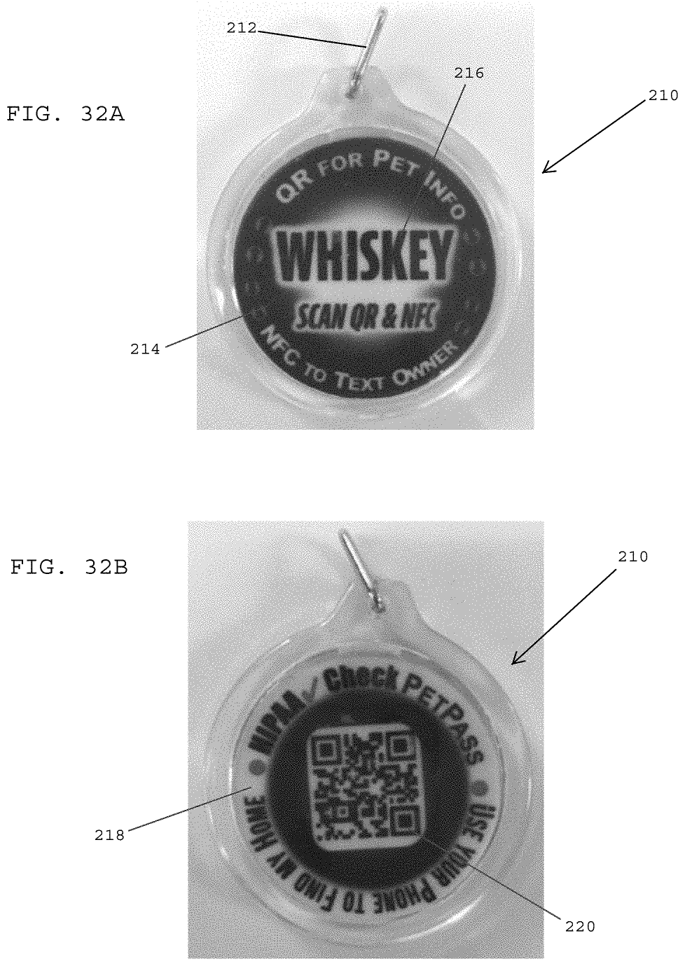

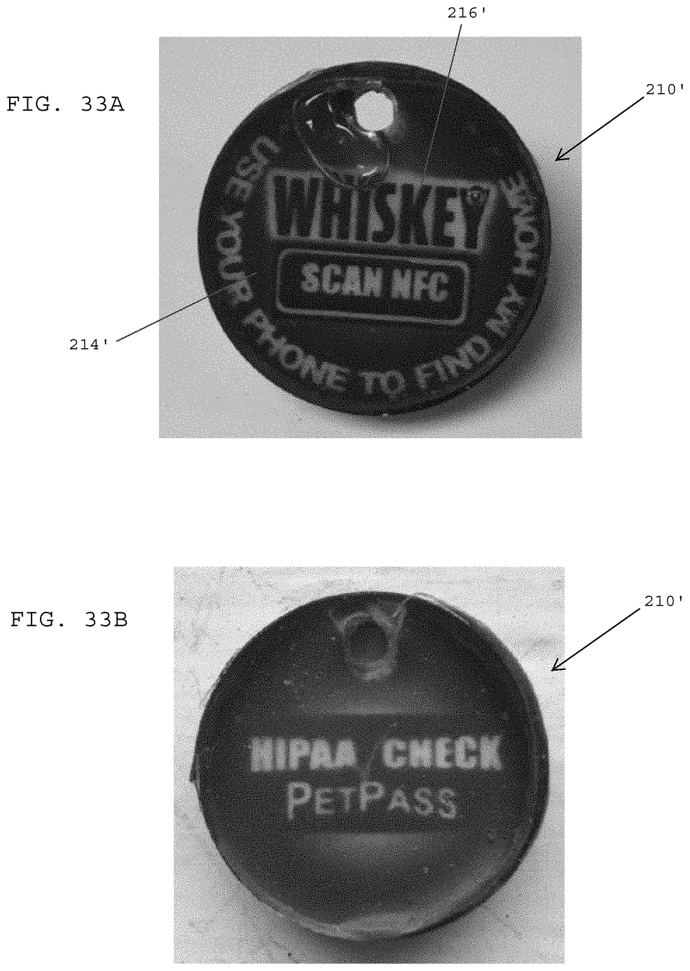

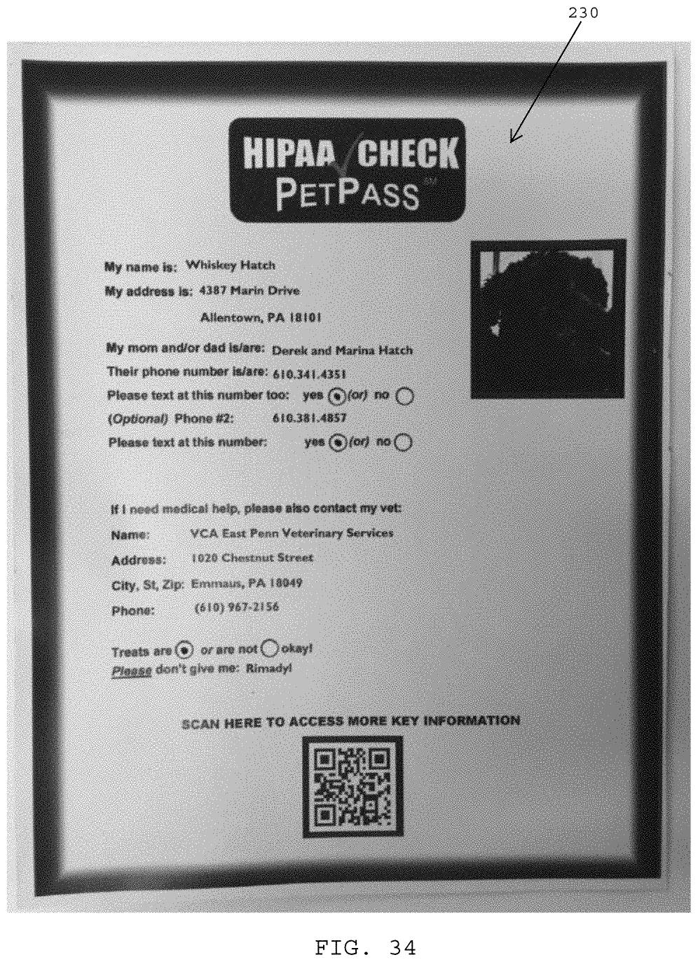

Referring to FIG. 34, in one embodiment, a system for identifying a lost or injured pet preferably includes a pet medical information sheet 230 that displays the information that was collected utilizing the pet medical information form 170 of FIG. 29, The pet medical information sheet 230 preferably includes identifying information about the pet, owner information for the pet, and veterinary information for the pet. The pet medical information sheet 230 also desirably includes a QR code 220 that is preferably the same as the QR code 220 provided on the pet tag 210 shown in FIG. 32B. A pet sitter or pet owner may scan the OR code 220 to access more identifying information or medical information about the pet associated with the pet medical information sheet 230.

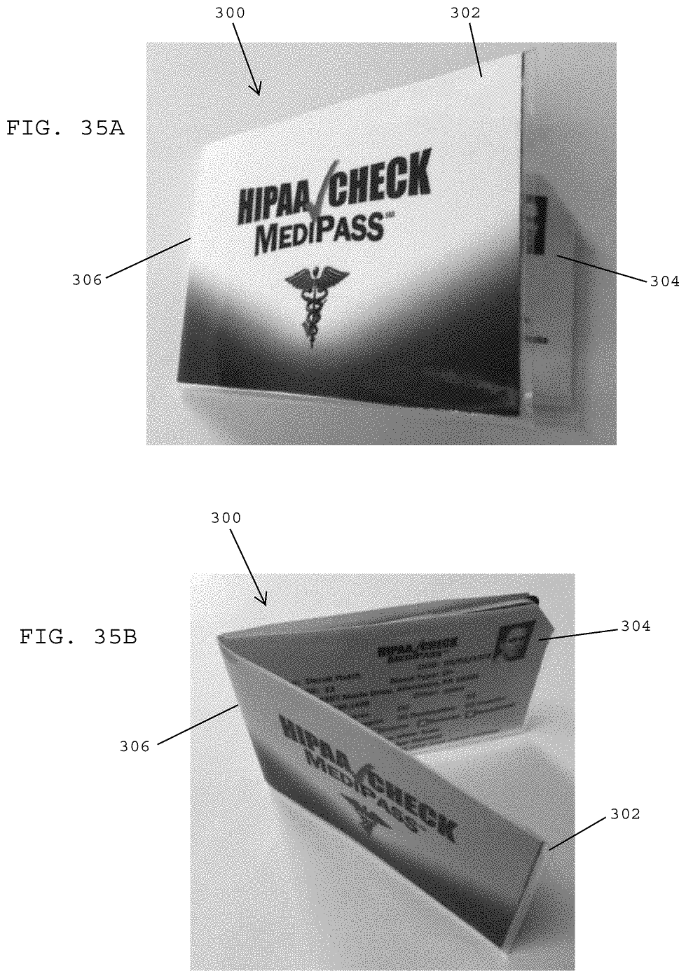

Referring to FIGS. 35A and 35B, in one embodiment, a protective sleeve 300 for a medical information passport preferably includes a first sleeve half 302 and a second sleeve half 304 that are joined together along a fold line 306.

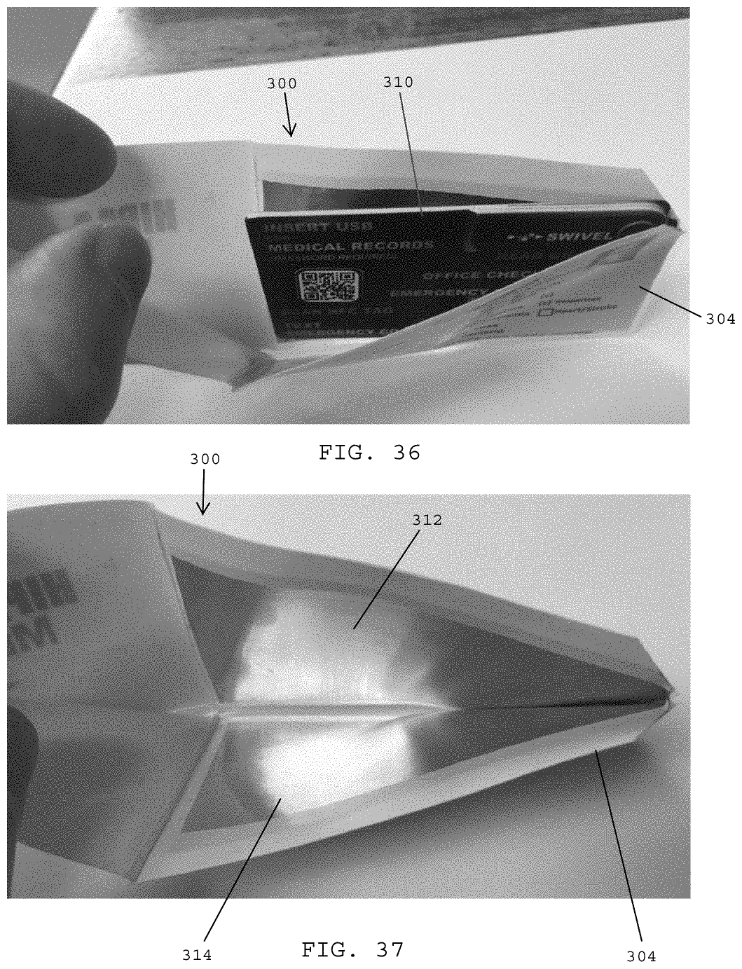

Referring to FIG. 36, in one embodiment, the protective sleeve 300 is configured to hold a medical information passport 310 that contains medical information for an individual, or that provides a link for accessing medical information associated with the individual. In one embodiment, the folder 300 preferably opens at an upper end of the second sleeve half 304 so that the medical information card 310 may be inserted into and withdrawn from the second sleeve half 304 of the protective sleeve 300.

Referring to FIGS. 36 and 37, in one embodiment, the inside surfaces of the second sleeve half 304 of the protective sleeve 300 are preferably covered with metal sheets 312, 314 that shield the medical information passport 310 when the medical information passport 310 is inserted into the opening of the second sleeve half 304 of the protective sleeve 300. The metal sheets 312, 314 preferably surround the medical information passport 310 so that the NFC tag may not be scanned by unauthorized personnel.

Referring to FIG. 38, in one embodiment, the protective sleeve 300 that holds the medical information passport 310 (FIG. 36) preferably includes the first sleeve half 302 and the second sleeve half 304 that are joined together along the fold line 306.

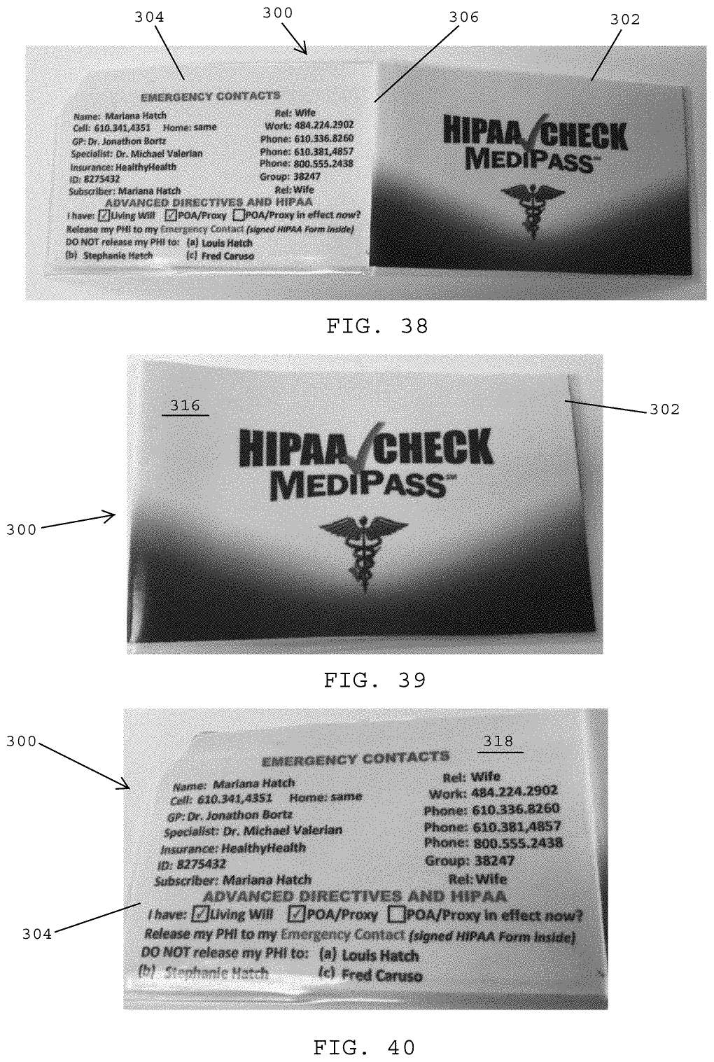

Referring to FIGS. 38 and 39, in one embodiment, the first sleeve half 302 of the protective sleeve 300 desirably has an outer face 316 that indicates that the protective sleeve 300 holds a medical information passport.

Referring to FIGS. 38 and 40, in one embodiment, the second sleeve half 304 of the protective sleeve 300 preferably has an outer face 318 that lists emergency contact information, advanced medical directives and HIPAA information that was collected using the forms 100 and 102 shown and described above in FIGS. 1-6.

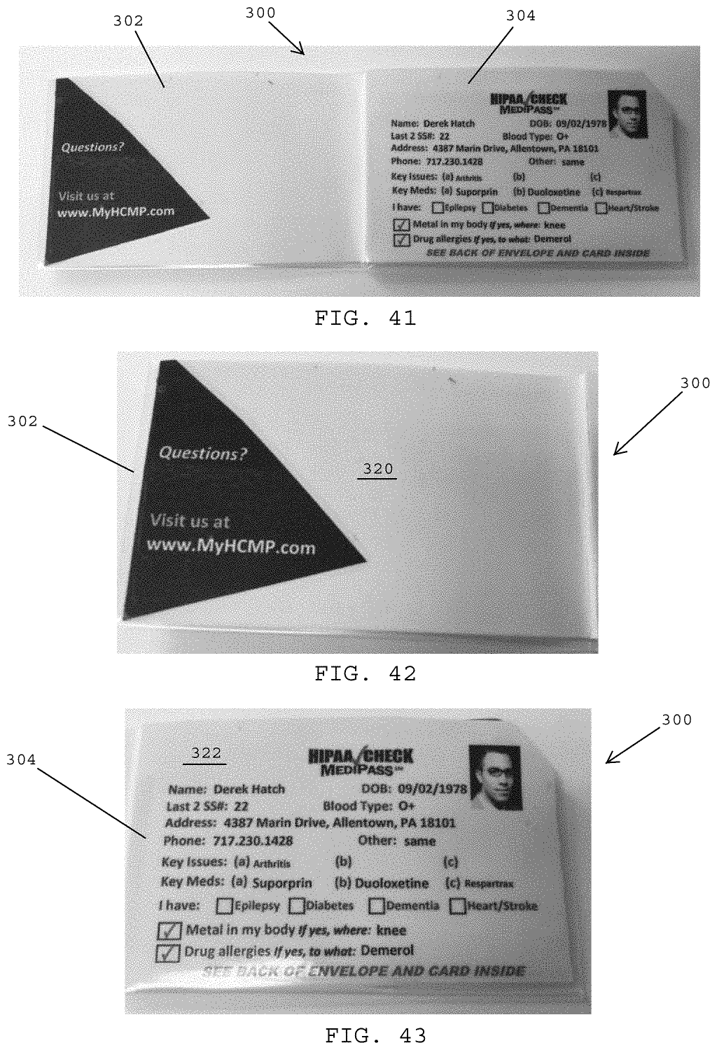

FIGS. 41-43 show the inner faces of the first and second sleeve halves 302, 304 of the protective sleeve 300. Referring to FIG. 42, the first sleeve half 302 of the protective sleeve 300 includes an inner face 320 that provides information regarding a link to a website containing medical information. Referring to FIG. 43, the second sleeve half 304 of the protective sleeve 300 has an inner face 322 that desirably contains additional identifying information about the individual carrying the medical information passport including identifying and medical information compiled utilizing the forms 100 and 102 shown and described above in FIGS. 1-6.

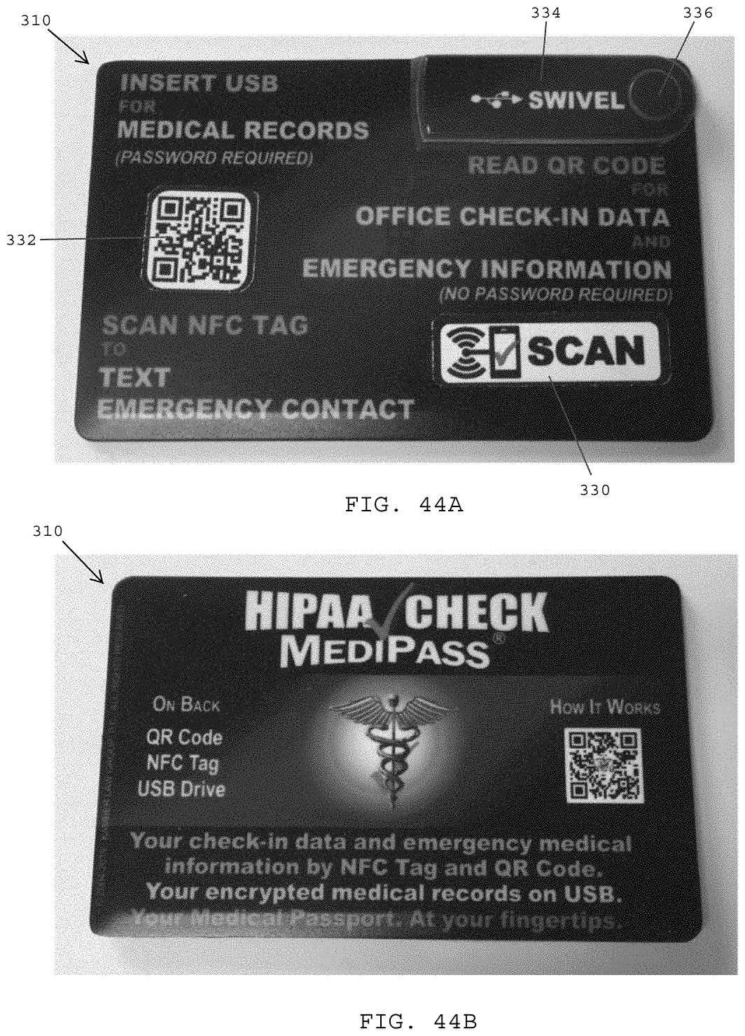

Referring to FIGS. 44A and 44B, in one embodiment, the medical information passport 310 may be utilized for sending text messages to emergency contacts, and accessing non-emergency and emergency medical information and medical records regarding an individual/patient. In one embodiment, the medical information passport 310 preferably includes an NFC tag 330 that may be scanned to send a text message to an emergency contact to indicate that the passport holder is incapacitated or undergoing emergency medical treatment. In one embodiment, the medical information passport 310 desirably includes a QR code 332 that may be scanned to obtain emergency medical information for an individual. In one embodiment, the medical information passport 310 preferably includes a USB flash drive 334 having a swivel mount 336 for enabling the USB flash drive 334 to swivel between a retracted position and an extended position for accessing medical information stored in the USB flash drive 334. In one embodiment, the USB flash drive 334 contains medical records and associated information that requires a password when being accessed. In one embodiment, the emergency contact information, the emergency medical information, and the detailed medical records requiring a password may all be linked and associated with one or more computer systems that may be accessed via the internet or a Cloud-based storage system, In one embodiment, all of the emergency contact, emergency medical, and detailed medical records are not stored on the medical information passport 310, but rather in a remote computer database. In one embodiment, the NFC tag, the QR code, and/or the USB flash drive may merely provide a means (e.g., links, hypertext) for accessing computer databases that contain the medical information.

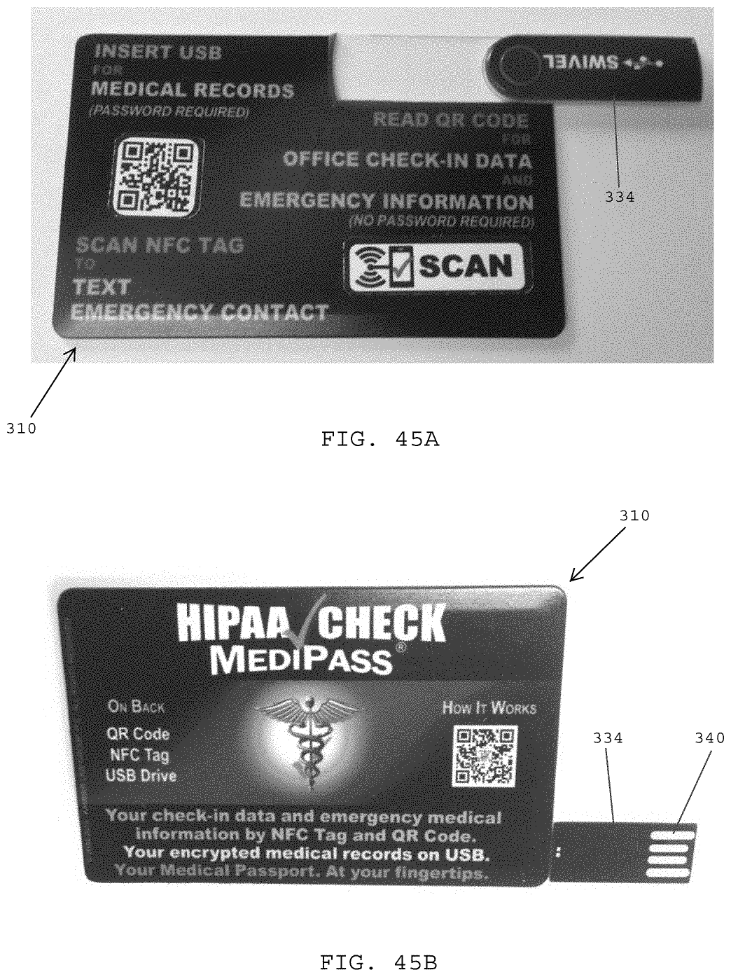

Referring to FIGS. 45A and 45B, in one embodiment, the USB flash drive 334 of the medical information passport 310 may be rotated to the extended position for exposing the USB contacts 340 so that the medical records of an individual may be accessed. In one embodiment, the USB contacts 340 are inserted into a USB port of a computer system for creating a link between the medical information passport 310 and the computer system containing the individual's detailed medical records. In one embodiment, a password is required to be authenticated before the detailed medical records of the individual are transmitted from the remote computer database to the computer coupled with the medical information passport 310.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

What is claimed is:

1. A system for providing medical information for a pet comprising:
a pet medical information form that contains medical information about a pet;
a pet medical information passport that includes identifying and medical information about the pet;
a pet tag worn by the pet, the pet tag including a QR code or a near field communication (NFC) device that stores emergency contact information for the pet and medical and insurance information for the pet.

2. The system as claimed in claim 1, wherein the NFC device on the pet tag stores emergency contact information including at least one telephone number for an emergency contact.

3. The system as claimed in claim 2, wherein the QR code links the pet medical information passport with a computer database having electronically stored emergency treatment information for the pet assigned to the pet medical information passport.

4. The system as claimed in claim 3, wherein the electronically stored emergency treatment information is not password protected.

5. The system as claimed in claim 3, wherein the electronically stored emergency treatment information is selected from the group of medical records consisting of MRI images, X-ray images, lab test results, doctor's notes, pharmacy records, insurance records, government records, surgical records, and any records provided for the pet assigned to the pet medical information passport.
